(19) United States
(12) Patent Application Publication
(10) Pub. No.: US 2010/0316626 A1
(43) Pub. Date: Dec. 16, 2010

(54) METHODS FOR TREATMENT AND DIAGNOSIS OF PSYCHIATRIC DISORDERS

(75) Inventor: Andres Buonanno, Bethesda, MD (US)

Correspondence Address: OTT- NIH;c/o WOODCOCK WASHBURN LLP, CIRA CENTRE, 12TH FLOOR, 2929 ARCH STREET, PHILADELPHIA, PA, 19104-2891, US

(73) Assignee: THE GOVERNMENT OF THE UNITED STATES OF AMERICA AS REPRESENTED BY THE SECRETARY, Rockville, MD (US)

(21) Appl. No.: 12/377,025

(22) PCT Filed: Aug. 10, 2007
(86) PCT No.: PCT/US07/75724

§ 371 Date: Jun. 14, 2010

Related U.S. Application Data
(60) Provisional application No. 60/837,449, filed on Aug. 11, 2006.

(51) Int. Cl. A61K 39/395; C12Q 1/68; G01N 33/53; A61K 38/00; A61K 31/7088; A61P 25/18; A61P 25/00; A61P 25/24

(52) U.S. Cl. 424/130.1; 435/6; 435/7.1; 514/1.1; 514/44 R; 514/44 A

(57) ABSTRACT

A method for preventing or treating a psychiatric disorder in a mammalian subject is provided. A method for preventing or treating a psychiatric disorder in a mammalian subject is provided which comprises administering to the subject a modulator of ErbB receptor signaling in an amount effective to reduce or eliminate the psychiatric disorder in the subject or to prevent its occurrence or recurrence. A method for diagnosis of a psychiatric disorder in a mammalian subject is provided.

### CROSS REFERENCE TO RELATED APPLICATION

This application claims the benefit of U.S. Provisional Application No. 60/837,449, filed Aug. 11, 2006, which is hereby incorporated by reference in its entirety.

### FIELD

The present invention relates to a method for preventing or treating a psychiatric disorder in a mammalian subject. The present invention provides a method for preventing or treating a psychiatric disorder in a mammalian subject comprising administering to the subject a modulator of ErbB receptor signaling in an amount effective to reduce or eliminate the psychiatric disorder in the subject or to prevent its occurrence or recurrence. The present invention relates to a method for diagnosis of a psychiatric disorder in a mammalian subject.

### BACKGROUND

Schizophrenia is a major psychiatric disorder affecting approximately 1% of the population. Although in most cases psychosis is manifested during the second decade of life (after puberty), there is an early developmental component to schizophrenia. All known typical and atypical antipsychotics used to treat schizophrenia work either as antagonist or partial agonists on D2-type dopamine receptors. These drugs mostly affect the positive symptoms of schizophrenia (psychosis) but are not as effective at treating the negative symptoms, which are thought to represent the endophenotype of the disorder. The most significant physiological problem with psychosis and cognition in schizophrenia, has been proposed to be the lack of differentiating meaningful from non-meaningful stimuli (or lack of affect) that may result from a general enhancement of cortical activity. Kwon et al., J. Neurosci, 25: 9378-9383, 2005; Lipska et al., Brain Res., 585: 1-6, 1992. A need exists in the art to develop novel strategies and pharmacological agents to treat both the positive and negative symptoms of schizophrenia, in particular the cognitive deficits which have the most significant impact on patients' lives. A need exists in the art to develop novel strategies and pharmacological agents to treat other psychiatric disorders, such as bi-polar depression, autism, and attention deficit disorder (ADD). The present invention is directed to this and other important ends.

### SUMMARY

The present invention generally relates to a method for preventing or treating a psychiatric disorder in a mammalian subject. The present invention provides a method for preventing or treating a psychiatric disorder in a mammalian subject comprising administering to the subject a modulator of ErbB receptor signaling in an amount effective to reduce or eliminate the psychiatric disorder in the subject or to prevent its occurrence or recurrence. The modulator can be an inhibitor of ErbB tyrosine kinase or an antagonist of ErbB receptor signaling pathway. The inhibitor or the antagonist can modulate induction or expression of long term potentiation in the mammalian subject. The modulator can be an activator of ErbB tyrosine kinase or an agonist of ErbB receptor signaling pathway. The activator or the agonist can modulate induction or expression of long term potentiation in the mammalian subject. The modulator can be a small chemical compound, an antisense RNA, an siRNA, shRNA, antibody, peptide or peptide mimetic. Treatment of the psychiatric disorder includes treatment for diseases, for example, schizophrenia, bipolar depression, autism or attention deficit disorder.

A method for treating a psychiatric disorder in a mammalian subject is provided which comprises administering to the subject a modulator of ErbB receptor signaling in an amount effective to reduce or eliminate the psychiatric disorder in the subject or to prevent its occurrence or recurrence. The ErbB receptor signaling can occur by ligand activation. The modulator can be an inhibitor of ErbB tyrosine kinase or an antagonist of ErbB receptor signaling pathway. The inhibitor or the antagonist can modulate induction or expression of long term potentiation in the mammalian subject. The modulator can be an activator of ErbB tyrosine kinase or an agonist of ErbB receptor signaling pathway. The activator or the agonist can modulate induction or expression of long term potentiation in the mammalian subject. The modulator can be a small chemical compound, an antisense RNA, an siRNA, shRNA, antibody, peptide or peptide mimetic. Treatment of the psychiatric disorder includes treatment for diseases, for example, schizophrenia, bipolar depression, autism or attention deficit disorder.

The method can further comprise administering to the subject a modulator of dopaminergic transmission signaling. The modulator of ErbB receptor signaling can affect an activity of dopamine D2 type receptor signaling. The modulator of dopaminergic transmission signaling can modulate induction or expression of long term potentiation in the mammalian subject. The modulator of ErbB receptor signaling can be a modulator of one or more ADAM proteases. The modulator of ErbB receptor signaling can be a modulator of gamma-secretase. The modulator of ErbB receptor signaling can be a modulator of glutamatergic transmission signaling. The ErbB receptor modulator can be a modulator of cholinergic transmission signaling. The method can further comprise administering to the subject a modulator of glutamatergic transmission signaling. The method can further comprise administering to the subject a modulator of dopaminergic transmission signaling. The method can further comprise administering to the subject a modulator of ADAM protease activity. The ErbB receptor includes, but is not limited to, ErbB1, ErbB2, ErbB3, or ErbB4. The ligand can be an EGF-like motif-containing ligand. The EGF-like motif-containing ligand can be neuregulin. The neuregulin includes, but is not limited to, neuregulin 1, neuregulin 2, neuregulin 3, or neuregulin 4. The modulator can include, but is not limited to, a small chemical compound, an antisense RNA, an siRNA, shRNA, antibody, peptide or peptide mimetic. The psychiatric disorder includes, but is not limited to, schizophrenia, bipolar depression, autism, or attention deficit disorder.

A method for identifying a modulator of signaling in cells via an ErbB receptor signaling pathway is provided which comprises contacting a test compound with a cell-based assay system comprising a cell expressing ErbB receptor capable of signaling responsiveness to a ligand and expressing dopamine receptor capable of signaling responsiveness to dopamine, providing the ligand to the assay system in an amount selected to be effective to activate ErbB receptor signaling, and detecting an effect of the test compound on ErbB receptor signaling in the assay system, effectiveness of the test compound in the assay being indicative of the modulation.

The ErbB receptor includes, but is not limited to, ErbB1, ErbB2, ErbB3, or ErbB4. The ligand can be an EGF-like motif-containing ligand. The EGF-like motif-containing ligand can be neuregulin. The neuregulin includes, but is not limited to, neuregulin 1, neuregulin 2, neuregulin 3, or neuregulin 4. The cells can behippocampal cells, neuronal cells, glial cells, brain tissue, olfactory epithelial cells, or neuroepithelial cells.

The method can further comprise the detecting step of measuring theta-pulse stimuli depotentiation of long term potentiation in the cell in response to ligand signaling via ErbB receptor. The detecting step can further comprise modulating depotentiation of long term potentiation in response to theta pulse stimuli by administration of the compound in the cellular assay. The detecting step can further comprise measuring dopamine receptor signaling in response to dopamine. The detecting step can further comprise measuring modulation of dopamine receptor signaling and modulation of long term potentiation in response to theta pulse stimuli by administration of the compound in the cellular assay. The method can further comprise screening a compound to treat a a psychiatric disorder in a mammalian subject by detecting an effect of the test compound on ErbB receptor signaling in the assay system, effectiveness of the test compound in the assay being indicative of efficacy of treatment in the mammalian subject.

A method for identifying a modulator of signaling in cells via an ErbB receptor signaling pathway is provided which comprises contacting a test compound with a tissue-based assay system comprising a tissue expressing ErbB receptor capable of signaling responsiveness to a ligand, providing the ligand to the assay system in an amount selected to be effective to modulate ErbB receptor signaling, and detecting an effect of the test compound on ErbB receptor signaling in the assay system by measuring a change in amplitude or frequency of gamma oscillatory activity, effectiveness of the test compound in the assay being indicative of the modulation. The detecting step further can further comprise measuring a change in frequency or power of kainate-induced or carbachol-induced gamma oscillatory activity. The tissue based assay can comprise brain tissue.

A method for screening a compound to treat a psychiatric disorder in a mammalian subject is provided which comprises contacting a test compound with a cell-based assay system comprising a cell expressing ErbB receptor capable of signaling responsiveness to a ligand and expressing D2 type receptor capable of signaling responsiveness to dopamine, providing the ligand to the assay system in an amount selected to be effective to modulate ErbB receptor signaling, and detecting an effect of the test compound on ErbB receptor signaling in the assay system, effectiveness of the test compound in the assay being indicative of efficacy of treatment in the mammalian subject. The psychiatric disorder includes, but is not limited to, schizophrenia, attention deficit disorder, bipolar depression, or autism.

A method for diagnosing a psychiatric disorder in a mammalian subject is provided which comprises isolating lymphocytes expressing NRG-1 gene and ErbB4 receptor, analyzing splice variants of NRG-1 mRNA or ErbB4 mRNA, determining a predisposition to a psychiatric disorder on the basis of splice variants of NRG-1 mRNA or ErbB4 mRNA. The NRG-1 splice variants can encode type I, type II, type III or type IV neuregulin. The psychiatric disorder includes, but is not limited to, schizophrenia, attention deficit disorder, bipolar depression, or autism.

A method for diagnosing a psychiatric disorder in a mammalian subject is provided which comprises isolating neuronal cells expressing NRG-1 gene and receptor ErbB4, analyzing polymorphisms of NRG-1 gene or ErbB4 gene, and determining a predisposition to a psychiatric disorder on the basis of polymorphisms of NRG-1 gene or ErbB4 gene. The polymorphism of NRG-1 gene or ErbB4 gene can be a single nucleotide polymorphism. The psychiatric disorder includes, but is not limited to, schizophrenia, attention deficit disorder, bipolar depression, or autism. The neuronal cell can be a hippocampal neuronal cell.

### BRIEF DESCRIPTION OF THE DRAWINGS

FIG. 1 shows chemical structure of small chemical ErbB inhibitors presently under clinical trials.

FIG. 2 shows core structures of quinalines (A) and pyridopyrimidines (B), and substitution sites for side groups that modify ErbB receptor preferences, affinity and reversibility of inhibitors.

FIG. 3 shows neuregulin-1β regulates LTP depotentiation.

FIG. 4 shows neuregulin-1β effects on LTP depotentiation are time dependent.

FIG. 5 shows neuregulin-1β selectively downregulates expression of surface AMPA receptors.

FIG. 6 shows neuregulin-1β causes internalization of surface AMPA receptors in dissociated hippocampal neurons.

FIG. 7 shows NRG-1 heterozygote mutant mice have enhanced LTP.

FIG. 8 shows NRG-1 effects on LTP in vivo are selectively mediated by ErbB-4 receptors.

FIG. 9 shows dopamine receptors couple to distinct G-couple pathways and have been implicated in LTP regulation.

FIG. 10 shows inhibition of the PI3K pathway prevents LTP depotentiation by NRG-1.

FIG. 11 shows blockade of D2-type receptors prevents NRG-1-dependent LTP depotentiation.

### DETAILED DESCRIPTION

### Overview

The present invention generally relates to a method for preventing or treating a psychiatric disorder in a mammalian subject. The present invention provides a method for preventing or treating a psychiatric disorder in a mammalian subject comprising administering to the subject a modulator of ErbB receptor signaling in an amount effective to reduce or eliminate the psychiatric disorder in the subject or to prevent its occurrence or recurrence. The modulator can be an antagonist of ErbB receptor signaling. The modulator can be an inhibitor of ErbB tyrosine kinase. The modulator of ErbB receptor signaling can also modulate dopaminergic transmission signaling. The modulator of dopaminergic transmission signaling can be an antagonist or inhibitor of dopamine D2 receptor signaling. The modulator of ErbB receptor signaling can also modulate glutamatergic transmission signaling. The modulator can be an antagonist or inhibitor of glutamate receptor signaling. The modulator of glutamatergic transmission signaling can be an antagonist or inhibitor of signaling through GABA receptor or an antagonist or inhibitor of signaling through Ach/acetylcholine receptor. The modulator of ErbB receptor signaling can be a small chemical compound, for example, an ADAM inhibitor or a gamma-secretase inhibitor. Treatment of the psychiatric disorder includes treatment for diseases, for example, schizophrenia, bipolar depression, or autism.

The present invention is based, in part, on the surprising observation that dopamine D2 receptor antagonists block the effect of neuregulin (also known as heregulin) on long term potentiation, the long lasting enhancement of synaptic transmission that, along with its counterpart, long term depression (LTD), is regarded as an important mechanism for memory formation and learning. This discovery of the connection between the neuregulin, its ErbB tyrosine kinase receptors, and the dopamine D2 receptor provides a basis for the treatment of schizophrenia using modulators of the ErbB signaling pathway, and in particular, inhibitors of the ErbB signaling pathway.

The neuregulins (NRGs) are a family of proteins containing an epidermal growth factor (EGF)-like motif that activates membrane-associated tyrosine kinases related to the EGF receptor (known as ErbB-1). Four genes encoding NRGs have been identified in vertebrates. NRG 1-4 have been found to bind preferentially to the ErbB-3-4 receptors, which then can form homo- or heterodimers by recruiting either the ErbB-1 or -2 receptors. Initially, NRG-1 was found to function in the nervous system in the proliferation of Schwann cells and in the regulation of nicotinic acetylcholine receptor transcription at the neuromuscular junction. The NRGs have also been found to regulate early fate determination, differentiation, migration and survival of satellite cells, Schwann cells and oligodendrocytes. In neurons, NRGs promote neuronal migration and selectively increase the expression of other neurotransmitter receptors.

Numerous studies have reported an association between neuregulin 1 and schizophrenia and have concluded that NRG-1 is a candidate gene for schizophrenia. Although it has been reported that schizophrenia may be caused by a defect in NRG-1/ErbB4 signaling that leads to decreased neurotransmission function, cross-talk between NRG/ErbB signaling and dopamine receptor signaling has been heretofore unknown. It has also been heretofore unknown that dopamine D2 receptor antagonists useful for the treatment of schizophrenia block the effect of neuregulin on long term potentiation and that agents that inhibit the NRG and ErbB signaling pathways can therefore be used to treat the disease.

The ErbB family of receptor tyrosine kinases, also known as the epidermal growth factor receptors, consist of four members including ErbB1 (also called epidermal growth factor receptor or Her1), ErbB2 (also called Her2 or neu), ErbB3 (also called Her3), and ErbB4 (also called Her4). The ErbB family of receptors are known to be important regulators of cell proliferation, differentiation, and survival and have been the targets of cancer drug discovery efforts. Neuregulin binds directly to ErbB4 and the present inventors have found the ErbB4 receptor to be essential for neurogelin signaling. After binding of its ligand, the ErbB4 receptor is cleaved by a metalloprotease. It has been found that the ErbB4 receptor is subsequently cleaved by a gamma secretase that releases the ErbB4 intracellular domain from the membrane and facilitates its translocation to the nucleus. (ni et al). After ligand binding, the ErbB receptors can trigger multiple downstream signaling cascades. (Zhou).

Category I inhibitors. Humanized monoclonal antibodies are presently in use which target the extracellular domains of ErbB-1 and -2 to promote their internalization and/or interfere with their dimerization. Perhaps the most successful examples are trastuzumab (trade name Herceptin) which is approved for treatment of breast cancers over-expressing ErbB-2, and cetuximab which is used to treat non-small-cell lung cancer (NSCLC); both are in on-going Phase I-IV clinical trials for combination treatments (Grunwald and Hidalgo, J. Natl. Cancer Inst. 95:851, 2003; Shridhar et al., Lancet Oncol. 4: 397, 2003; Abanel and Cascon, 2005; Shelton et al., Expert Opin. Ther. Targets 9: 1009, 2005). Other humanized antibodies targeting ErbB-1 and -2 are at different stages of on-going clinical trials.

ErbB inhibitors for use in the treatment methods of the present invention include modulators of the ErbB pathway, such as antibodies directed against the ErBb receptors, including, trastuzumab (Herceptin™, Genentech), cetuximab (Erbutix™, ImClone Systems), ABX-EGF (Amgen), EMD-7200 (EMD pharma), Pertuzumab (Omnitarg™, Genentech).

Category II inhibitor. Herstatin (previously known as Dimercept) is a naturally occurring soluble, truncated extracellular portion of ErbB-2 protein that can bind to ErbB receptors to prevent their dimerization and signaling with other ErbB receptors.

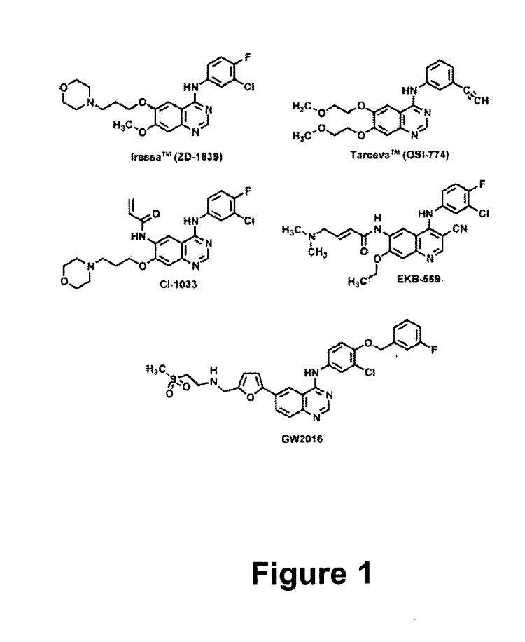

Category III inhibitors. Category I and II approaches show promise for treating a variety of cancers in peripheral tissues, but their use to treat psychiatric disorders will require that they cross the blood-brain barrier either by specific modifications to the protein or by finding methods to make the blood-brain barrier permeable to these type of reagents. The small, membrane permeable molecules with distinct preferences for ErbB receptors are compatible as systemically delivered compounds that may cross the blood-brain barrier. FIG. 1 shows chemical structure of small chemical ErbB inhibitors presently under clinical trials (from Fry, Exp. Cell Research 284: 131, 2003). A variety of these small organic ErbB inhibitors are presently in different stages of preclinical and clinical trials for the treatment of non-small cell lung carcinomas, adenocarcinoma, breast cancer and glioblastomas (see FIG. 1). Among these inhibitors are gefitinib (ZD1839, Iressa) and erlotinib (Tarceva, CP-358774, OSI 774) which have preferences for ErbB1 receptors, and ones with a broader ErbB receptor specificity which include lapatinib (GW572016), EKB-569, CI-1033 (PD183805), AEE788, PKI-146, and PD158780 (rev. Shridhar et al., Lancet Oncol. 4: 397, 2003; Shelton et al., Expert Opin. Ther. Targets 9: 1009, 2005). This application focuses on the Category III compounds.

Small molecule tyrosine kinase inhibitors such as GW572016 (Lapatinib, Glaxo SmithKline), ZD-1839 or gefitinib (Iressa™, AstraZeneca Pharmaceuticals), erlotinib (Tarceva™, Genentech), CI-1033 (Pfizer), CP-654577 (Pfizer), Arry-334543 (ArrayBioPharma), PKI-166 (Novartis), AEE788 (Novartis), PD158780, PD160678, PD168393, PD160879, PD174265. Fry et al., Experimental Cell Research, 284: 131-139, 2003; Molina et al., Esteva The Oncologist, Barbacci et al, Wood et al., Chu et al. 6-substituted 4-anilinoquinazolines and 4-anilinopyrido[3,4-d]pyrimidines are provided in Klutchko et al., J. Med. Chem., 49: 1475-1485, 2006.

ErbB signaling is mediated by metalloprotease cleavage, more specifically by cleavage by members of the ADAM family of metalloproteases. The present invention provides methods for treatment of psychiatric disorders, for example, schizophrenia or attention deficit disorder, by administering an inhibitor of an ADAM metalloproteases and thereby modulating the ErbB receptor signaling. ADAM inhibitors for use in the methods for treatment of psychiatric disorders of the present invention include inhibitors of the ADAM metalloproteases that are involved in ErbB signaling, such as ADAM17, ADAM12, and ADAM10. (Yan et al, Zhou, Ann N.Y. Acad. Sci., 1059: 56-60, 2005.

ErbB receptor signaling has also been found to be mediated by gamma secretases (Ni et al). The present invention provides methods for treatment of psychiatric disorders by administering an inhibitor of gamma secretase. The inhibitors modulate signaling in the ErbB pathway by inhibiting cleaving of ErbB receptors by gamma secretase.

Gamma secretase inhibitors for use in the methods for treatment of psychiatric disorders of the present invention include, but are not limited to, L-685,458 (L1790) (Sigma-Aldrich, Inc.), S1120 (A.G. Scientific, Inc.), sulindac sulfide (SSide), DAPT (Boehringer Ingelheim Pharma KG; Dovey H. F. et al. J. Neurochem., 76: 173-181, 2001.), LY450139 dihydrate, or gamma secretase inhibitors from Calbiochem, Inc. Some gamma secretase inhibitors are non-competitive inhibitors.

Current treatment regimens for schizophrenia are directed toward the administration of antagonist or partial agonists of D2-type domain receptors to subjects in need thereof. Antipsychotics currently in use for the treatment of schizophrenia include, for example, aripiprazole (Abilify™), clozapine (Clozaril™), zisprasidone (Geodon™), haloperidol (Haldol™), molindone (Lindone™), loxapine (Loxitane™), thioridazine (Mellaril™), molindone (Moban™), thiothixene (Navane™), pimozide (Orap™), fluphenazine (Permitil™), fluphenazine (Prolixin™), risperidone (Risperdal™), mesoridazine (Serentil™), quetiapine (Seroquel™) trifluoperazine (Stelazine™), chlorprothixene (Taractan™), chlorpromazine (Thorazine™), perphenazine (Trilafon™), trifluoprmazine (Vesprin Trifluopromazine™), and olanzapine (Zyprexa™).

The methods for treatment of a psychiatric disorder in a mammalian subject of the present invention provide for administration of a modulator of the ErbB pathway to the mammalian subject. In one aspect the modulator of the ErbB pathway is an antagonist or inhibitor of the ErbB pathway. The methods of the present invention further provide for the concomitant administration of an antagonist or partial agonists of dopamine D2-type domain receptors with a modulator of the ErbB pathway. “Concomitant administration” of a antagonist or partial agonists of dopamine D2-type domain receptors with a modulator of the ErbB pathway means administration of the two agents at such time that both the agents will have a therapeutic effect on the psychiatric disorder in the mammalian subject. Such concomitant administration can involve concurrent (i.e., at the same time), prior, or subsequent administration of the antagonist or partial agonists of dopamine D2-type domain receptors with respect to the administration of a modulator of the ErbB pathway.

It is to be understood that this invention is not limited to particular methods, reagents, compounds compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Modulators”, “inhibitors,” and “activators,” of ErbB receptor signaling and/or dopaminergic transmission signaling, e.g., dopamine D2 type receptors, or glutamatergic transmission signaling via NRG-1 or glutamate are used to refer to inhibitory, activating, or modulating molecules, respectively, identified using in vitro and in vivo assays for NRG-1 or glutamate binding or signaling, e.g., ligands, agonists, antagonists, and their homologs and mimetics. In general, methods for treating a psychiatric disorder in a mammalian subject comprise administering an antagonist or inhibitor of ErbB receptor signaling and/or dopaminergic transmission signaling, e.g., dopamine D2 type receptors, or glutamatergic transmission signaling. In some cases combinations of modulators, e.g., inhibitors and activators, can be used to treat the psychiatric disorder. “Modulator” includes inhibitors, activators, antagonists, and agonists. Inhibitors are agents that, e.g., bind to, partially or totally block stimulation, decrease, prevent, delay activation, inactivate, desensitize, or down regulate the activity of ErbB receptor signaling and/or dopaminergic transmission signaling, or glutamatergic transmission signaling, e.g., antagonists. Activators are agents that, e.g., bind to, stimulate, increase, open, activate, facilitate, enhance activation, sensitize or up regulate the activity of ErbB receptor signaling and/or dopaminergic transmission signaling, or glutamatergic transmission signaling, e.g., agonists. Modulators include agents that, e.g., alter the interaction of ErbB receptor, dopamine receptor, or glutamate receptor with: proteins that bind activators or inhibitors, receptors, including proteins, peptides, lipids, carbohydrates, polysaccharides, or combinations of the above, e.g., lipoproteins, glycoproteins. Modulators include genetically modified versions of naturally-occurring ErbB receptor, dopamine receptor, or glutamate receptor ligands, e.g., NRG-1 or glutamate, with altered activity, as well as naturally occurring and synthetic ligands, antagonists, agonists, small chemical molecules. Such assays for inhibitors and activators include, e.g., applying putative modulator compounds to a cell expressing NRG-1 and then determining the functional effects ErbB receptor signaling and/or dopaminergic transmission signaling, e.g., dopamine D2 type receptors, or glutamatergic transmission signaling, as described herein. Samples or assays comprising NRG-1 or glutamate that are treated with a potential activator, inhibitor, or modulator are compared to control samples without the inhibitor, activator, or modulator to examine the extent of inhibition. Control samples (untreated with inhibitors) can be assigned a relative ErbB receptor, dopamine receptor, or glutamate receptor activity value of 100%. Inhibition of ErbB receptor signaling and/or dopaminergic transmission signaling, or glutamatergic transmission signaling is achieved when the ErbB receptor, dopamine receptor, or glutamate receptor activity value relative to the control is about 80%, optionally 50% or 25-0%. Activation of ErbB receptor, dopamine receptor, or glutamate receptor is achieved when the ErbB receptor, dopamine receptor, or glutamate receptor activity value relative to the control is 110%, optionally 150%, optionally 200-500%, or 1000-3000% higher.

“Antagonist” is used in the broadest sense, and includes any molecule that partially or fully blocks, inhibits, or neutralizes a biological activity of a ErbB receptor signaling and/or dopaminergic transmission signaling, or glutamatergic transmission signaling or NRG-1 polypeptide activity. In a similar manner, the term “agonist” is used in the broadest sense and includes any molecule that mimics or enhances a biological activity of a ErbB receptor signaling and/or dopaminergic transmission signaling, or glutamatergic transmission signaling. Suitable agonist or antagonist molecules specifically include agonist or antagonist antibodies or antibody fragments, fragments or amino acid sequence variants of native NRG-1 polypeptides, peptides, glutamate or analogs thereof, antisense oligonucleotides, small organic molecules. Methods for identifying agonists or antagonists of a ErbB receptor, dopamine receptor, or glutamate receptor can comprise contacting a ErbB receptor, dopamine receptor, or glutamate receptor polypeptide with a candidate agonist or antagonist molecule and measuring a detectable change in one or more biological activities normally associated with the ErbB receptor, dopamine receptor, or glutamate receptor polypeptide.

“A modulator of the Neuregulin (NRG)-ErbB receptor signaling pathway” refers to those molecules, e.g., organic, biological or a modified biological molecule, that have the capacity to either augment or decrease the signaling potential of ErbB receptors, e.g., inhibitors, activators, antagonists, or agonists. These include, but are not limited to: reagents that either increase or decrease the availability of ligands to bind ErbB receptors to modulate ErbB receptor activity; molecules in the brain, peripheral tissues or circulating in blood that modify the levels of ErbB ligands; molecules that either increase or decrease the surface levels of functional ErbB receptors; and molecules that enhance or interfere with the downstream signaling pathways activated by ErbB receptor activity.

The rationale for proposing modulators of ErbB receptors to treat psychiatric disorders is based on evidence from research in primates demonstrating that the dose-response curve of neurotransmitters (i.e., dopamine) and working memory is an inverted U-shape, as shown in the figure below.

The figure shows the relationship between dopamine levels and working memory performance is an inverted U-shape curve. The relative levels of dopamine are represented in the abscissa and working memory on the ordinate. A) As relative dopamine levels increase, working memory performance improves to a maximum and then begins to fall as dopamine levels continue to augment. B) Performance of subjects with basal dopamine levels that are not optimal (red; left of curve) can be improved with treatments that increase its levels (green; top of curve); for example, by inhibiting COMT, an enzyme that degrades liberated dopamine. C) However, in subjects performing at optimal levels (green; top of curve) treatment with COMT inhibitors, which result in higher dopamine levels, causes a relative decrease in working memory performance (red; right of curve). Therefore, it is plausible that either activators or inhibitors of the ErbB signaling pathway could lead to improvements in patients with psychiatric disorders.

“Cell-based assays” include ErbB receptor, dopamine receptor, or glutamate receptor binding assays, for example, radioligand or fluorescent ligand binding assays for ErbB receptor signaling and/or dopaminergic transmission signaling, or glutamatergic transmission signaling to cells, plasma membranes, detergent-solubilized plasma membrane proteins, immobilized collagen (Alberdi, J Biol Chem. 274:31605-12, 1999; Meyer et al., 2002); ErbB receptor signaling-affinity column chromatography (Alberdi, J Biol Chem. 274:31605-12, 1999; Aymerich et al., Invest Opthalmol Vis Sci. 42:3287-93, 2001); ErbB receptor-ligand blot using a radio- or fluorosceinated-ligand (Aymerich et al., Invest Opthalmol Vis Sci. 42:3287-93, 2001; Meyer et al., 2002); Size-exclusion ultrafiltration (Alberdi et al., Biochem., 1998; Meyer et al., 2002); or ELISA. Exemplary ErbB receptor binding activity assays of the present invention are: a NRG-1 or glutamate/ErbB receptor ligand blot assay (Aymerich et al., Invest Opthalmol Vis Sci. 42:3287-93, 2001); a ErbB receptor affinity column chromatography assay (Alberdi, J Biol Chem. 274:31605-12, 1999) and a NRG-1 or glutamate/ErbB receptor ligand binding assay (Alberdi et al., J Biol Chem. 274:31605-12, 1999). Each incorporated by reference in their entirety.

“Contacting” refers to mixing a test compound in a soluble form into an assay system, for example, a cell-based assay system, such that an effect upon receptor-mediated signaling can be measured.

“Signaling in cells” refers to the interaction of a ligand, such as an endogenous or exogenous ligand, e.g., NRG-1 or glutamate, with receptors, such as ErbB receptor signaling and/or dopaminergic transmission signaling, or glutamatergic transmission signaling resulting in cell signaling to produce a response, for example, treatment of a psychiatric disorder, e.g., schizophrenia or attention deficit disorder. “Signaling responsiveness” or “effective to activate signaling” or “stimulating a cell-based assay system” refers to the ability of ErbB receptor signaling and/or dopaminergic transmission signaling, e.g., dopamine D2 type receptors, or glutamatergic transmission to signal via NRG-1 or glutamate to treat a psychiatric disorder, e.g., schizophrenia or attention deficit disorder.

“Test compound” refers to a small chemical entity nucleic acid, DNA, RNA, protein, or polypeptide, that is determined to effect an increase or decrease in signaling through the ErbB receptor and/or dopaminergic transmission, or glutamatergic transmission pathway. The term “test compound” can be any small chemical compound, or a biological entity, such as a protein, sugar, nucleic acid or lipid. Typically, test compounds will be small chemical molecules and polypeptides. A “test compound specific for signaling by ErbB receptor and/or dopaminergic transmission signaling, or glutamatergic transmission signaling” is determined to be a modulator of ErbB receptor signaling and/or dopaminergic transmission signaling, or glutamatergic transmission signaling via NRG-1 or glutamate.

“Detecting an effect” refers to an effect measured in a cell-based assay system. For example, the effect detected can be ErbB receptor signaling and/or dopaminergic transmission signaling, or glutamatergic transmission signaling in an assay system, for example, NRG-1 or glutamate cellular assay, ErbB receptor, dopamine receptor, or glutamate receptor binding assay.

“Assay being indicative of modulation” refers to results of a cell-based assay system indicating that cell activation by ErbB receptor, dopamine receptor, or glutamate receptor signaling via NRG-1 or glutamate induces a protective response in cells to treat a psychiatric disorder, e.g., schizophrenia or attention deficit disorder.

“Biological activity” and “biologically active” with regard to a ligand of ErbB receptor signaling and/or dopaminergic transmission signaling, or glutamatergic transmission signaling of the present invention refer to the ability of the ligand molecule to specifically bind to and signal through a native or recombinant ErbB receptor, dopamine receptor, or glutamate receptor, or to block the ability of a native or recombinant ErbB receptor, dopamine receptor, or glutamate receptor to participate in signal transduction. Thus, the (native and variant) ligands of ErbB receptor, dopamine receptor, or glutamate receptor of the present invention include agonists and antagonists of a native or recombinant ErbB receptor, dopamine receptor, or glutamate receptor. Preferred biological activities of the ligands of ErbB receptor signaling and/or dopaminergic transmission signaling, or glutamatergic transmission signaling of the present invention include the ability to to treat a psychiatric disorder, e.g., schizophrenia or attention deficit disorder. Accordingly, the administration of the compounds or agents of the present invention can prevent or delay, to alleviate, or to arrest or inhibit development of the symptoms or conditions associated with a psychiatric disorder, e.g., schizophrenia or attention deficit disorder.

“High affinity” for a ligand refers to an equilibrium association constant (Ka) of at least about 10³M⁻¹, at least about 10⁴ M⁻¹, at least about 10⁵ M⁻¹, at least about 10⁶ M⁻¹, at least about 10⁷ M⁻¹, at least about 10⁸ M⁻¹, at least about 10⁹ M⁻¹, at least about 10¹⁰ M⁻¹, at least about 10¹¹ M⁻¹, or at least about 10¹² M⁻¹ or greater, e.g., up to 10¹³ M⁻¹ or 10¹⁴ M⁻¹ or greater. However, “high affinity” binding can vary for other ligands.

“Kₐ”, as used herein, is intended to refer to the equilibrium association constant of a particular ligand-receptor interaction, e.g., antibody-antigen interaction. This constant has units of 1/M.

“K_{d}”, as used herein, is intended to refer to the equilibrium dissociation constant of a particular ligand-receptor interaction. This constant has units of M.

“kₐ”, as used herein, is intended to refer to the kinetic association constant of a particular ligand-receptor interaction. This constant has units of 1/Ms.

“k_{d}”, as used herein, is intended to refer to the kinetic dissociation constant of a particular ligand-receptor interaction. This constant has units of 1/s.

“Particular ligand-receptor interactions” refers to the experimental conditions under which the equilibrium and kinetic constants are measured.

“Isotype” refers to the antibody class that is encoded by heavy chain constant region genes. Heavy chains are classified as gamma, mu, alpha, delta, or epsilon, and define the antibody's isotype as IgG, IgM, IgA, IgD and IgE, respectively. Additional structural variations characterize distinct subtypes of IgG (e.g., IgG₁, IgG₂, IgG₃ and IgG₄) and IgA (e.g., IgA₁ and IgA₂)

“Sorting” in the context of cells as used herein to refers to both physical sorting of the cells, as can be accomplished using, e.g., a fluorescence activated cell sorter, as well as to analysis of cells based on expression of cell surface markers, e.g., FACS analysis in the absence of sorting.

“Cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny cannot be precisely identical in DNA content, due to deliberate or inadvertent mutations. Mutant progeny that have the same function or biological activity as screened for in the originally transformed cell are included. Where distinct designations are intended, it will be clear from the context.

“Patient”, “subject” or “mammalian subject” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles. “Mammalian subject” or “patient” refers to any mammalian patient or subject to which the compositions of the invention can be administered. The term mammals, human patients and non-human primates, as well as experimental animals such as rabbits, rats, and mice, and other animals. In an exemplary embodiment, of the present invention, to identify subject patients for treatment according to the methods of the invention, accepted screening methods are employed to determine risk factors associated with a targeted or suspected disease or condition, e.g., a psychiatric disorder, or to determine the status of an existing disease or condition in a subject. These screening methods include, for example, conventional work-ups to determine risk factors that can be associated with the targeted or suspected disease or condition. These and other routine methods allow the clinician to select patients in need of therapy using the methods and formulations of the invention.

“Psychiatric disorder” refers to psychiatric diseases, including but not limited to, schizophrenia, bipolar disorder, autism and attention deficit disorders

“Treating” or “treatment” includes the administration of the compositions, compounds or agents of the present invention to prevent or delay the onset of the symptoms, complications, or biochemical indicia of a disease, alleviating or ameliorating the symptoms or arresting or inhibiting further development of the disease, condition, or disorder (e.g., a psychiatric disorder). “Treating” further refers to any indicia of success in the treatment or amelioration or prevention of the disease, condition, or disorder (e.g., a psychiatric disorder), including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with a psychiatric disorder. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of a psychiatric disorder but does not yet experience or exhibit symptoms, inhibiting the symptoms of a psychiatric disorder (slowing or arresting its development), providing relief from the symptoms or side-effects of psychiatric disorder (including palliative treatment), and relieving the symptoms of psychiatric disorder (causing regression of disease). Treatment can be prophylactic (to prevent or delay the onset of the disease, or to prevent the manifestation of clinical or subclinical symptoms thereof) or therapeutic suppression or alleviation of symptoms after the manifestation of the disease or condition.

“Concomitant administration” of a known drug with a compound of the present invention means administration of the drug and the compound at such time that both the known drug and the compound will have a therapeutic effect or diagnostic effect. Such concomitant administration can involve concurrent (i.e. at the same time), prior, or subsequent administration of the drug with respect to the administration of a compound of the present invention. A person of ordinary skill in the art, would have no difficulty determining the appropriate timing, sequence and dosages of administration for particular drugs and compounds of the present invention.

In general, the phrase “well tolerated” refers to the absence of adverse changes in health status that occur as a result of the treatment and would affect treatment decisions.

The ability of a molecule to bind to ErbB receptor tyrosine kinase, dopamine D2 receptor, or glutamate receptor can be determined, for example, by the ability of the putative ligand to bind to ErbB receptor tyrosine kinase immunoadhesin coated on an assay plate. Specificity of binding can be determined by comparing binding to non-ErbB receptor tyrosine kinase immunoadhesin.

In one embodiment, ligand binding to ErbB receptor tyrosine kinase can be assayed by either immobilizing the ligand or the receptor. For example, the assay can include immobilizing ErbB receptor tyrosine kinase fused to a His tag onto Ni-activated NTA resin beads. Antibody can be added in an appropriate buffer and the beads incubated for a period of time at a given temperature. After washes to remove unbound material, the bound protein can be released with, for example, SDS, buffers with a high pH, and analyzed.

“Epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

“Substantially pure” or “isolated” means an object species (e.g., a receptor or ligand of the invention) has been identified and separated and/or recovered from a component of its natural environment such that the object species is the predominant species present (i.e., on a molar basis it is more abundant than any other individual species in the composition); a “substantially pure” or “isolated” composition also means where the object species comprises at least about 50 percent (on a molar basis) of all macromolecular species present. A substantially pure or isolated composition can also comprise more than about 80 to 90 percent by weight of all macromolecular species present in the composition. An isolated object species (e.g., receptors or ligands of the invention) can also be purified to essential homogeneity (contaminant species cannot be detected in the composition by conventional detection methods) wherein the composition consists essentially of derivatives of a single macromolecular species. For example, an isolated ligand to ErbB receptor tyrosine kinase can be substantially free of other ligands that lack binding to human ErbB receptor tyrosine kinase and bind to a different receptor. Further, an isolated ligand that specifically binds to an epitope, isoform or variant of human ErbB receptor tyrosine kinase may, however, have cross-reactivity to other related ligand, e.g., from other species (e.g., ErbB receptor tyrosine kinase species homologs). Moreover, an isolated ligand of the invention be substantially free of other cellular material and/or chemicals.

“Naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

“Signal transduction pathway” or “signal transduction event” refers to at least one biochemical reaction, but more commonly a series of biochemical reactions, which result from interaction of a cell with a stimulatory compound or agent, e.g., ErbB receptor signaling and/or dopaminergic transmission signaling, e.g., dopamine D2 type receptors, or glutamatergic transmission signaling via NRG-1 or glutamate. Thus, the interaction of a stimulatory compound with a cell generates a “signal” that is transmitted through the signal transduction pathway, ultimately resulting in a cellular response, e.g., a decrease in symptoms of a psychiatric disorder, as described above.

A signal transduction pathway refers to the biochemical relationship between a variety of signal transduction molecules that play a role in the transmission of a signal from one portion of a cell to another portion of a cell. As used herein, the phrase “cell surface receptor” includes molecules and complexes of molecules capable of receiving a signal and the transmission of such a signal across the plasma membrane of a cell. An example of a “cell surface receptor” is the ErbB receptor, dopamine D2 receptor, or glutamate receptor which determine ErbB receptor signaling and/or dopaminergic transmission signaling, or glutamatergic transmission signaling.

A signal transduction pathway in a cell can be initiated by interaction of a cell with a stimulator that is inside or outside of the cell. If an exterior (i.e., outside of the cell) stimulator (e.g., NRG-1 or glutamate) interacts with a cell surface receptor (e.g., a ErbB receptor, dopamine receptor, or glutamate receptor), a signal transduction pathway can transmit a signal across the cell's membrane, through the cytoplasm of the cell, and in some instances into the nucleus. If an interior (e.g., inside the cell) stimulator interacts with an intracellular signal transduction molecule, a signal transduction pathway can result in transmission of a signal through the cell's cytoplasm, and in some instances into the cell's nucleus.

Signal transduction can occur through, e.g., the phosphorylation of a molecule; non-covalent allosteric interactions; complexing of molecules; change of protein localization; the conformational change of a molecule; calcium release; inositol phosphate production; proteolytic cleavage; cyclic nucleotide production and diacylglyceride production. Typically, signal transduction occurs through phosphorylating a signal transduction molecule.

“Receptor” denotes a cell-associated protein, for example, ErbB receptor, dopamine receptor, or glutamate receptor, that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., ErbB receptor, TNF receptor I, TNF receptor II, PDGF receptor, growth hormone receptor, IL-3 receptor, GM-CSF receptor, G-CSF receptor, erythropoietin receptor and IL-6 receptor). Membrane-bound receptors, for example ErbB receptor, dopamine D2 receptor, or glutamate receptor, are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

In general, the binding of ligand to receptor results in a conformational change in the receptor that causes an interaction between the effector domain and other molecule(s) in the cell, which in turn leads to an alteration in the metabolism of the cell. Metabolic events that are often linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids.

By “solid phase” is meant a non-aqueous matrix to which a reagent of interest (e.g., ErbB receptor, dopamine receptor, or glutamate receptor or a ligand binding to the receptor) can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

“Specifically (or selectively) binds” to a receptor refers to a binding reaction that is determinative of the presence of a ligand and/or receptor in a heterogeneous population of proteins and other biologics. Thus, under designated immunoassay conditions, the specified antibodies bind to a particular protein at least two times the background and do not substantially bind in a significant amount to other proteins present in the sample. Similarly, ligand binding to a receptor can be measured as at least two times the background and do not substantially bind in a significant amount to other ligands or receptors present in the sample. “Specifically bind(s)” or “bind(s) specifically” when referring to a peptide refers to a peptide molecule which has intermediate or high binding affinity, exclusively or predominately, to a target molecule. The phrase “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA immunoassays, immunoprecipitation, Biacore and Western blot are used to identify ligands, e.g., NRG-1, peptides, or small chemical molecule inhibitors that specifically react with ErbB receptor, dopamine receptor, or glutamate receptor domain-containing proteins. Typically a specific or selective reaction will be at least twice background signal or noise and more typically more than 10 times background. Specific binding between a monovalent ligand and ErbB receptor, dopamine receptor, or glutamate receptor means a binding affinity of at least 10³ M⁻¹, and preferably 10⁵, 10⁶, 10⁷, 10⁸, 10⁹ or 10¹⁰ M⁻¹. The binding affinity of NRG-1 to ErbB receptor is between about 10⁶ M⁻¹ to about 10¹⁰M⁻¹.

The present invention is based on the discovery that the ErbB receptor, dopamine receptor, or glutamate receptor is a specific sensor of endogenous and exogenous ligands which are necessary for signaling via the ErbB receptor, dopamine receptor, or glutamate receptor pathway. NRG-1 protein or glutamate binds ErbB receptor, dopamine receptor, or glutamate receptor with a high affinity. The experiments described herein demonstrate that ErbB receptor, dopamine receptor, or glutamate receptor is a receptor which can play a role in mediating responses to NRG-1 or glutamate. In particular, the ErbB receptor and native NRG-1 polypeptides, peptides, glutamate or analogs thereof has been found to be present in a variety of tissue and cell populations, for example, hippocampal neurons, and are necessary for treating a psychiatric disorder, e.g., schizophrenia or attention deficit disorder.

This invention relies on routine techniques in the field of recombinant genetics. Basic texts disclosing the general methods of use in this invention include Sambrook et al., Molecular Cloning, A Laboratory Manual, 2nd ed., 1989; Kriegler, Gene Transfer and Expression: A Laboratory Manual, 1990; and Ausubel et al., eds., Current Protocols in Molecular Biology, 1994.

ErbB receptor, dopamine receptor, or glutamate receptor nucleic acids, polymorphic variants, orthologs, and alleles that are substantially identical to sequences provided herein can be isolated using ErbB receptor, dopamine receptor, or glutamate receptor nucleic acid probes and oligonucleotides under stringent hybridization conditions, by screening libraries. Alternatively, expression libraries can be used to clone ErbB receptor, dopamine receptor, or glutamate receptor protein, polymorphic variants, orthologs, and alleles by detecting expressed homologs immunologically with antisera or purified antibodies made against human ErbB receptor, dopamine receptor, or glutamate receptor, or portions thereof.

“Nucleic acid” or “nucleic acid molecule” refer to a deoxyribonucleotide or ribonucleotide polymer in either single- or double-stranded form, and unless otherwise limited, can encompass known analogs of natural nucleotides that can function in a similar manner as naturally occurring nucleotides.

“Isolated nucleic acid” in reference to nucleic acids encoding ErbB receptor tyrosine kinase, is intended to refer to a nucleic acid in which the nucleotide sequences encoding the antibody or antibody portion are free of other nucleotide sequences encoding antibodies or antibody portions that bind antigens other than, for example, ErbB receptor tyrosine kinase, which other sequences can naturally flank the nucleic acid in human genomic DNA.

“Substantially identical,” in the context of two nucleic acids or polypeptides refers to two or more sequences or subsequences that have at least about 80%, about 90%, about 95% or higher nucleotide or amino acid residue identity, when compared and aligned for maximum correspondence, as measured using the following sequence comparison method and/or by visual inspection. Such “substantially identical” sequences are typically considered to be homologous. The “substantial identity” can exist over a region of sequence that is at least about 50 residues in length, over a region of at least about 100 residues, or over a region at least about 150 residues, or over the full length of the two sequences to be compared. As described below, any two antibody sequences can only be aligned in one way, by using the numbering scheme in Kabat. Therefore, for antibodies, percent identity has a unique and well-defined meaning.

ErbB receptor tyrosine kinase, dopamine receptor, or glutamate receptor, nucleic acids, polymorphic variants, orthologs, and alleles that are substantially identical to sequences provided herein can be isolated using nucleic acid probes and oligonucleotides of ErbB receptor, dopamine receptor, or glutamate receptor, under stringent hybridization conditions, by screening libraries. Alternatively, expression libraries can be used to isolate ErbB receptor tyrosine kinase protein, or protein encoding ErbB receptor tyrosine kinase polymorphic variants, orthologs, and alleles by detecting expressed homologs immunologically with antisera or purified antibodies made against human ErbB receptor tyrosine kinase, or portions thereof.

The nucleic acids of the invention be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art (See, e.g., Sambrook et al., Molecular Cloning, A Laboratory Manual, 2^{nd} ed., 1989; Tijssen (1993); and Ausubel (1994), incorporated by reference for all purposes). The nucleic acid sequences of the invention and other nucleic acids used to practice this invention, whether RNA, cDNA, genomic DNA, or hybrids thereof, can be isolated from a variety of sources, genetically engineered, amplified, and/or expressed recombinantly. Any recombinant expression system can be used, including, in addition to bacterial, e.g., yeast, insect or mammalian systems. Alternatively, these nucleic acids can be chemically synthesized in vitro. Techniques for the manipulation of nucleic acids, such as, e.g., subcloning into expression vectors, labeling probes, sequencing, and hybridization are well described in the scientific and patent literature, see, e.g., Sambrook, et al., 1989. Nucleic acids can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, radioimmunoassay (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

The nucleic acid compositions of the present invention, while often in a native sequence (except for modified restriction sites), from either cDNA, genomic or mixtures can be mutated, thereof in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

“Recombinant host cell” or “host cell” refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

“Control sequences” or “regulatory sequences” refers to DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

“Vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available (e.g., the polypeptides of the invention can be made detectable, e.g., by incorporating a radiolabel into the peptide, and used to detect antibodies specifically reactive with the peptide).

Components of an immune response can be detected in vitro by various methods that are well known to those of ordinary skill in the art. For example, (1) cytotoxic T lymphocytes can be incubated with radioactively labeled target cells and the lysis of these target cells detected by the release of radioactivity, (2) helper T lymphocytes can be incubated with antigens and antigen presenting cells and the synthesis and secretion of cytokines and proliferation as measured by assays described below and measured by standard methods (Windhagen A; et al., Immunity 2:373-380, 1995), (3) antigen presenting cells can be incubated with whole protein antigen and the presentation of that antigen on MHC detected by either T lymphocyte activation assays or biophysical methods (Harding et al., Proc. Natl. Acad. Sci. U.S.A., 86:4230-4, 1989), (4) mast cells can be incubated with reagents that cross-link their Fc-epsilon receptors and histamine release measured by enzyme immunoassay (Siraganian, et al., TIPS 4:432-437, 1983).

Similarly, products of an immune response in either a model organism (e.g., mouse) or a human patient can also be detected by various methods that are well known to those of ordinary skill in the art. For example, (1) the production of antibodies in response to vaccination can be readily detected by standard methods currently used in clinical laboratories, e.g., an ELISA; (2) the migration of immune cells to sites of inflammation can be detected by scratching the surface of skin and placing a sterile container to capture the migrating cells over scratch site (Peters et al., Blood 72:1310-5, 1988); (3) the proliferation of peripheral blood mononuclear cells in response to mitogens or mixed lymphocyte reaction can be measured using ³H-thymidine; (4) the phagocitic capacity of granulocytes, macrophages, and other phagocytes in PBMCs can be measured by placing PMBCs in wells together with labeled particles (Peters et al., Blood 72:1310-5, 1988); and (5) the radioimmunoassay of immune system cells can be measured by labeling PBMCs with antibodies to CD molecules such as CD4 and CD8 and measuring the fraction of the PBMCs expressing these markers.

“Immune response” refers to the concerted action of lymphocytes, antigen presenting cells, phagocytic cells, granulocytes, and soluble macromolecules produced by the above cells (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues. “Immune cell response” refers to the response of immune system cells to external or internal stimuli (e.g., antigen, cytokines, chemokines, and other cells) producing biochemical changes in the immune cells that result in immune cell migration, killing of target cells, phagocytosis, production of antibodies, other soluble effectors of the immune response.

“T lymphocyte response” and “T lymphocyte activity” are used here interchangeably to refer to the component of immune response dependent on T lymphocytes (i.e., the proliferation and/or differentiation of T lymphocytes into helper, cytotoxic killer, or suppressor T lymphocytes, the provision of signals by helper T lymphocytes to B lymphocytes that cause or prevent antibody production, the killing of specific target cells by cytotoxic T lymphocytes, and the release of soluble factors such as cytokines that modulate the function of other immune cells).

“Antibody” is used in the broadest sense and specifically covers monoclonal antibodies, antibody compositions with polyepitopic specificity, bispecific antibodies, diabodies, and single-chain molecules, as well as antibody fragments (e.g., Fab, F(ab′)₂, and Fv), so long as they exhibit the desired biological activity. Antibodies can be labeled/conjugated to toxic or non-toxic moieties. Toxic moieties include, for example, bacterial toxins, viral toxins, radioisotopes. Antibodies can be labeled for use in biological assays (e.g., radioisotope labels, fluorescent labels) to aid in detection of the antibody. Antibodies can also be labeled/conjugated for diagnostic or therapeutic purposes, e.g., with radioactive isotopes that deliver radiation directly to a desired site for applications such as radioimmunotherapy (Garmestani. et al., Nucl Med Biol, 28:409, 2001), imaging techniques and radioimmunoguided surgery or labels that allow for in vivo imaging or detection of specific antibody/antigen complexes. Antibodies can also be conjugated with toxins to provide an immunotoxin (see, Kreitman, R J Adv Drug Del Rev, 31:53, 1998).

An intact “antibody” comprises at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H}1 C_{H}2 and C_{H}3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) through cellular receptors such as Fc receptors (e.g., FcγRI, FcγRIIa, FcγRIIb, FcγRIII, and FcRrη) and the first component (Clq) of the classical complement system. The term antibody includes antigen-binding portions of an intact antibody that retain capacity to bind the antigen. Examples of antigen binding portions include (i) a Fab fragment, a monovalent fragment consisting of the V_{L}, V_{H}, CL and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); See, e.g., Bird et al., Science 242: 423-426, 1988; and Huston et al., Proc. Natl. Acad. Sci. U.S.A. 85: 5879-5883, 1988). Such single chain antibodies are included by reference to the term “antibody” Fragments can be prepared by recombinant techniques or enzymatic or chemical cleavage of intact antibodies.

“Human sequence antibody” includes antibodies having variable and constant regions (if present) derived from human immunoglobulin sequences. The human sequence antibodies can include amino acid residues not encoded by human germline immunoglobulin sequences (e.g., mutations introduced by random or site-specific mutagenesis in vitro or by somatic mutation in vivo). However, the term “human sequence antibody”, as used herein, is not intended to include antibodies in which entire CDR sequences sufficient to confer antigen specificity and derived from the germline of another mammalian species, such as a mouse, have been grafted onto human framework sequences (i.e., humanized antibodies).

“Monoclonal antibody” refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method first described by Kohler et al., Nature, 256: 495, 1975, or can be made by recombinant DNA methods (see, e.g., U.S. Pat. No. 4,816,567, Cabilly et al.). The “monoclonal antibodies” can also be isolated from phage antibody libraries using the techniques described in Clackson et al., 624-628, 1991, and Marks et al., J. Mol. Biol. 222:581-597, 1991, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Cabilly et al., supra; Morrison et al., Proc. Natl. Acad. Sci. USA, 81:6851-6855, 1984.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature 321:522-525, 1986; Reichmann et al., Nature 332:323-329, 1988; and Presta, Curr. Op. Struct. Biol. 2:593-596, 1992. The humanized antibody includes a Primatized™ antibody wherein the antigen-binding region of the antibody is derived from an antibody produced by immunizing macaque monkeys with the antigen of interest.

Amino acids from the variable regions of the mature heavy and light chains of immunoglobulins are designated Hx and Lx respectively, where x is a number designating the position of an amino acids according to the scheme of Kabat et al., 1987 and 1991, Sequences of Proteins of Immunological Interest (National Institutes of Health, Bethesda, Md.). Kabat et al. list many amino acid sequences for antibodies for each subclass, and list the most commonly occurring amino acid for each residue position in that subclass. Kabat et al. use a method for assigning a residue number to each amino acid in a listed sequence, and this method for assigning residue numbers has become standard in the field. The scheme of Kabat et al. is extendible to other antibodies not included in the compendium by aligning the antibody in question with one of the consensus sequences in Kabat et al. The use of the Kabat et al. numbering system readily identifies amino acids at equivalent positions in different antibodies. For example, an amino acid at the L50 position of a human antibody occupies the equivalence position to an amino acid position L50 of a mouse antibody

“Non-immunogenic in a human” means that upon contacting the polypeptide of interest in a physiologically acceptable carrier and in a therapeutically effective amount with the appropriate tissue of a human, no state of sensitivity or resistance to the polypeptide of interest is demonstrable upon the second administration of the polypeptide of interest after an appropriate latent period (e.g., 8 to 14 days).

“Neutralizing antibody” refers to an antibody which is able to block or significantly reduce an effector function of wild type or mutant NRG-1 polypeptides, peptides, glutamate or analogs thereof or glutamate or analogs thereof on ErbB receptor, dopamine receptor, or glutamate receptor signaling. For example, a neutralizing antibody can inhibit or reduce ErbB receptor inhibition by an antagonist antibody, as determined, for example, in an NRG-1/ErbB receptor binding assay, or other assays taught herein or known in the art.

### Identification of Compounds for Treatment and Prophylaxis of Disease

The present invention provides a method for preventing or treating a psychiatric disorder, e.g., schizophrenia or attention deficit disorder, in a mammalian subject comprises administering to the mammalian subject a compound capable of inhibiting a ErbB receptor tyrosine kinase, wherein the compound is administered in an amount effective to reduce or eliminate the psychiatric disorder or to prevent its occurrence or recurrence. The inhibitor of ErbB receptor signaling can be, for example, a small chemical compound. The present invention further provides a method for identifying a compound capable of preventing or treating psychiatric disorder comprising contacting a test compound with a cell-based assay system comprising a cell expressing ErbB tyrosine kinase and capable of signaling responsiveness to ErbB tyrosine kinase, and detecting an effect of the test compound on ErbB tyrosine kinase signaling in the assay system as a decrease in susceptibility of the cell line to ErbB receptor signaling and/or dopaminergic transmission signaling, or glutamatergic transmission signaling, effectiveness of the test compound in the assay being indicative of the effective treatment of a psychiatric disorder.

Identifying bioactive agents that modulate ErbB receptor signaling and/or dopaminergic transmission signaling, e.g., dopamine D2 type receptors, or glutamatergic transmission signaling via NRG-1 or glutamate can be performed in a wide variety of ways. In one method, one of several cellular assays, e.g., ErbB receptor, dopamine receptor, or glutamate receptor binding assays, can be used in conjunction with high throughput screening techniques, to allow monitoring for modulators of ErbB receptor signaling and/or dopaminergic transmission signaling, or glutamatergic transmission signaling after treatment with a candidate agent, Zlokarnik, et al., Science 279:84-8, 1998; and Heid et al., Genome Res. 6:986, 1996; each incorporated herein by reference in their entirety. In one method, the candidate agents are added to cells.

“Candidate bioactive agent” or “drug candidate” or grammatical equivalents as used herein describes any molecule, e.g., protein, oligopeptide, small organic molecule, polysaccharide, polynucleotide, to be tested for bioactive agents that are capable of directly or indirectly altering the activity of ErbB receptor signaling and/or dopaminergic transmission signaling, or glutamatergic transmission signaling. In one method, the bioactive agents modulate ErbB receptor signaling and/or dopaminergic transmission signaling, e.g., dopamine D2 type receptors, or glutamatergic transmission signaling via NRG-1 or glutamate. In a further embodiment of the method, the candidate agents induce a modulator, antagonist, or agonist effect in a ErbB receptor signaling and/or dopaminergic transmission signaling, or glutamatergic transmission signaling binding assay, as further described below. Generally a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents can be subjected to directed or random chemical modifications, such as acylation, alkylation, esterification, amidification to produce structural analogs.

In some embodiments, the candidate bioactive agents are proteins. By “protein” herein is meant at least two covalently attached amino acids, which includes proteins, polypeptides, oligopeptides and peptides. The protein can be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the methods herein. “Amino acid” also includes imino acid residues such as proline and hydroxyproline. The side chains can be in either the (R) or the (S) configuration. In further embodiments, the amino acids are in the (S) or (L)-configuration. If non-naturally occurring side chains are used, non-amino acid substituents can be used, for example to prevent or retard in vivo degradations.

In one method, the candidate bioactive agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and mammalian proteins, and human proteins.

In some methods, the candidate bioactive agents are peptides of from about 5 to about 30 amino acids, typically from about 5 to about 20 amino acids, and typically from about 7 to about 15 being. The peptides can be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they can incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In some methods, the library can be fully randomized, with no sequence preferences or constants at any position. In other methods, the library can be biased. Some positions within the sequence are either held constant, or are selected from a limited number of possibilities. For example, in some methods, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, or to purines. In other methods, the candidate bioactive agents are nucleic acids, as defined above.

As described above generally for proteins, nucleic acid candidate bioactive agents can be naturally occurring nucleic acids, random nucleic acids, or “biased” random nucleic acids. For example, digests of procaryotic or eucaryotic genomes can be used as is outlined above for proteins.

In some methods, the candidate bioactive agents are organic chemical moieties.

(A) Drug Screening Methods

Several different drug screening methods can be accomplished to identify drugs or bioactive agents that modulate ErbB receptor signaling and/or dopaminergic transmission signaling, e.g., dopamine D2 type receptors, or glutamatergic transmission signaling via NRG-1 or glutamate. One such method is the screening of candidate agents that can act as an antagonist of ErbB receptor, dopamine receptor, or glutamate receptor, thus generating the associated phenotype. Candidate agents that can act as an antagonist to ErbB receptor signaling and/or dopaminergic transmission signaling, or glutamatergic transmission signaling, as shown herein, are expected to result in a phenotype indicating a decrease in a psychiatric related disorder. Thus, in some methods, candidate agents can be determined that mimic or alter ErbB receptor pathway signaling.

In other methods, screening can be done to alter the biological function of the ErbB receptor signaling and/or dopaminergic transmission signaling, or glutamatergic transmission signaling. Again, having identified the importance of NRG-1 or glutamate in ErbB receptor signaling and/or dopaminergic transmission signaling, or glutamatergic transmission signaling, screening for agents that bind and/or modulate the biological activity of the ErbB receptor, dopamine receptor, or glutamate receptor can be performed as outlined below.

Thus, screening of candidate agents that modulate ErbB receptor pathway signaling and/or dopaminergic transmission pathway signaling, or glutamatergic transmission pathway signaling pathway either at the level of gene expression or protein level can be accomplished.

In some methods, a candidate agent can be administered in any one of several cellular assays, e.g., ErbB receptor, dopamine receptor, or glutamate receptor binding assay. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) can be put into a viral construct such as a retroviral construct and added to the cell, such that expression of the peptide agent is accomplished; see PCT US97/01019, incorporated herein by reference in its entirety.

Once the candidate agent has been administered to the cells, the cells can be washed if desired and are allowed to incubate under physiological conditions for some period of time. The cells are then harvested and a new gene expression profile is generated, as outlined herein.

For example, ErbB receptor signaling and/or dopaminergic transmission signaling, e.g., dopamine D2 type receptors, or glutamatergic transmission signaling via NRG-1 or glutamate can be screened for agents that produce a phenotype indicating a decrease in a psychiatric-related disorder. A change in a binding assay or cellular assay upon addition of the candidate agent indicates that the agent has an affect on ErbB receptor, dopamine receptor, or glutamate receptor pathway signaling activity, e.g., an inhibitor of receptor activity. By defining such a signature for treating a psychiatric disorder, screens for new drugs that alter receptor mediated activity and indicate a decrease in a psychiatric related disorder can be devised. With this approach, the drug target need not be known and need not be represented in the original expression screening platform, nor does the level of transcript for the target protein need to change. In some methods, the agent acts as an antagonist or agonist in one of several cellular or binding assays, e.g., ErbB receptor, dopamine receptor, or glutamate receptor binding assay.

In some methods, screens can be done on individual genes and gene products. After having identified a cellular or binding assay as indicative of an antipsychotic treatment for a psychiatric disorder, screening of modulators of cellular or binding assay can be completed.

Thus, in some methods, screening for modulators of cellular or binding assay can be completed. This will be done as outlined above, but in general a few cellular or binding assay are evaluated. In some methods, screens are designed to first find candidate agents that can affect a cellular activity or binding assay, and then these agents can be used in other assays that evaluate the ability of the candidate agent to modulate ErbB receptor signaling and/or dopaminergic transmission signaling, or glutamatergic transmission signaling.

In general, purified or isolated gene product can be used for binding assays; that is, the gene products of ErbB receptor, dopamine receptor, or glutamate receptor are made. Using the nucleic acids of the methods and compositions herein which encode a ErbB receptor, dopamine receptor, or glutamate receptor, a variety of expression vectors can be made. The expression vectors can be either self-replicating extrachromosomal vectors or vectors which integrate into a host genome. Generally, these expression vectors include transcriptional and translational regulatory nucleic acid operably linked to the nucleic acid encoding a ErbB receptor, dopamine receptor, or glutamate receptor or NRG-1 protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express ErbB receptor, dopamine receptor, glutamate receptor, or NRG-1 protein; for example, transcriptional and translational regulatory nucleic acid sequences from Bacillus are used to express the protein in Bacillus. Numerous types of appropriate expression vectors, and suitable regulatory sequences are known in the art for a variety of host cells.

In general, the transcriptional and translational regulatory sequences can include, but are not limited to, promoter sequences, ribosomal binding sites, transcriptional start and stop sequences, translational start and stop sequences, and enhancer or activator sequences. In one method, the regulatory sequences include a promoter and transcriptional start and stop sequences.

Promoter sequences encode either constitutive or inducible promoters. The promoters can be either naturally occurring promoters or hybrid promoters. Hybrid promoters, which combine elements of more than one promoter, are also known in the art, and are useful in the methods herein.

In addition, the expression vector can comprise additional elements. For example, the expression vector can have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and typically two homologous sequences which flank the expression construct. The integrating vector can be directed to a specific locus in the host cell by selecting the appropriate homologous sequence for inclusion in the vector. Constructs for integrating vectors are well known in the art. Methods to effect homologous recombination are described in PCT US93/03868 and PCT US98/05223, each incorporated herein by reference in their entirety.

In some methods, the expression vector contains a selectable marker gene to allow the selection of transformed host cells. Selection genes are well known in the art and will vary with the host cell used.

One expression vector system is a retroviral vector system such as is generally described in PCT/US97/01019 and PCT/US97/01048, each incorporated herein by reference in their entirety.

The ErbB receptor, dopamine receptor, glutamate receptor or NRG-1 proteins of the present methods and compositions are produced by culturing a host cell transformed with an expression vector containing nucleic acid encoding NRG-1 or ErbB receptor, under the appropriate conditions to induce or cause expression of the protein. The conditions appropriate for NRG-1 or ErbB receptor expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In some methods, the timing of the harvest is important. For example, the baculoviral systems used in insect cell expression are lytic viruses, and thus harvest time selection can be crucial for product yield.

Appropriate host cells include yeast, bacteria, archebacteria, fungi, and insect and animal cells, including mammalian cells. Of particular interest are Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells. In some methods, brain hippocampal neurons are host cells as provided herein, which for example, include non-recombinant cell lines, such as primary cell lines. In addition, purified ErbB receptor, dopamine receptor, glutamate receptor, or NRG-1 for receptor assay derived from either transgenic or non-transgenic strains can also be used. The host cell can alternatively be an cell type known to have immunodeficiency disorder.

In one method, the ErbB receptor, dopamine receptor, glutamate receptor, or NRG-1 proteins can be expressed in mammalian cells. Mammalian expression systems can include retroviral systems. A mammalian promoter is any DNA sequence capable of binding mammalian RNA polymerase and initiating the downstream (3′) transcription of a coding sequence for ErbB receptor, dopamine receptor, glutamate receptor, or NRG-1 proteins into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In some methods, ErbB receptor, dopamine receptor, glutamate receptor, or NRG-1 proteins are expressed in bacterial systems which are well known in the art.

In other methods, ErbB receptor, dopamine receptor, glutamate receptor, or NRG-1 proteins can be produced in insect cells. Expression vectors for the transformation of insect cells, and in particular, baculovirus-based expression vectors, are well known in the art.

In some methods, ErbB receptor, dopamine receptor, glutamate receptor, or NRG-1 proteins are produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

A ErbB receptor, dopamine receptor, glutamate receptor, or NRG-1 protein can also be made as a fusion protein, using techniques well known in the art. For example, for the creation of monoclonal antibodies, if the desired epitope is small, the protein can be fused to a carrier protein to form an immunogen. Alternatively, ErbB receptor, dopamine receptor, glutamate receptor, or NRG-1 proteins can be made as a fusion protein to increase expression. For example, when a protein is a shorter peptide, the nucleic acid encoding the peptide can be linked to other nucleic acid for expression purposes. Similarly, ErbB receptor, dopamine receptor, glutamate receptor, or NRG-1 proteins of the methods and compositions herein can be linked to protein labels, such as green fluorescent protein (GFP), red fluorescent protein (RFP), yellow fluorescent protein (YFP), and blue fluorescent protein (BFP).

In one embodiment, the proteins are recombinant. A “recombinant protein” is a protein made using recombinant techniques, i.e., through the expression of a recombinant nucleic acid as depicted above. A recombinant protein is distinguished from naturally occurring protein by at least one or more characteristics. For example, the protein can be isolated or purified away from some or all of the proteins and compounds with which it is normally associated in its wild type host, and thus can be substantially pure. For example, an isolated protein is unaccompanied by at least some of the material with which it is normally associated in its natural state, typically constituting at least about 0.5%, typically at least about 5% by weight of the total protein in a given sample. A substantially pure protein comprises at least about 75% by weight of the total protein, at least about 80%, and typically at least about 90%. The definition includes the production of ErbB receptor, dopamine receptor, glutamate receptor, or NRG-1 proteins from one organism in a different organism or host cell. Alternatively, the protein can be made at a significantly higher concentration than is normally seen, through the use of a inducible promoter or high expression promoter, such that the protein is made at increased concentration levels. Alternatively, the protein can be in a form not normally found in nature, as in the addition of an epitope tag or amino acid substitutions, insertions and deletions, as discussed below.

In some methods, when the ErbB receptor, dopamine receptor, glutamate receptor, or NRG-1 proteins is to be used to generate antibodies, the protein must share at least one epitope or determinant with the full length transcription product of the nucleic acids. By “epitope” or “determinant” herein is meant a portion of a protein which will bind an antibody. Thus, in most instances, antibodies made to a smaller protein should be able to bind to the full length protein. In one embodiment, the epitope is unique; that is, antibodies generated to a unique epitope show little or no cross-reactivity.

In some methods, the antibodies provided herein can be capable of reducing or eliminating the biological function of a ErbB receptor, dopamine receptor, glutamate receptor, or NRG-1 protein, as is described below. The addition of antibodies (either polyclonal or monoclonal) to the protein (or cells containing the protein) can reduce or eliminate the protein's activity. Generally, at least a 25% decrease in activity is observed, with typically at least about 50% and typically about a 95-100% decrease being observed.

In addition, the proteins can be variant proteins, comprising one more amino acid substitutions, insertions and deletions.

In one method, a ErbB receptor, dopamine receptor, glutamate receptor, or NRG-1 protein is purified or isolated after expression. Proteins can be isolated or purified in a variety of ways. Standard purification methods include electrophoretic, molecular, immunological and chromatographic techniques, including ion exchange, hydrophobic, affinity, and reverse-phase HPLC chromatography, and chromatofocusing. For example, a ErbB receptor, dopamine receptor, glutamate receptor, or NRG-1 protein can be purified using a standard anti-NRG-1 or anti-ErbB receptor antibody column. Ultrafiltration and diafiltration techniques, in conjunction with protein concentration, are also useful. For general guidance in suitable purification techniques, see Scopes, Protein Purification, Springer-Verlag, N.Y., 1982, incorporated herein by reference in its entirety. The degree of purification necessary will vary depending on the use of the protein. In some instances no purification will be necessary.

Once the gene product of the ErbB receptor, dopamine receptor, glutamate receptor, or NRG-1 gene is made, binding assays can be done. These methods comprise combining an ErbB receptor, dopamine receptor, glutamate receptor, or NRG-1 protein and a candidate bioactive agent, and determining the binding of the candidate agent to the ErbB receptor, dopamine receptor, glutamate receptor, or NRG-1 protein. Methods utilize a human ErbB receptor, dopamine receptor, glutamate receptor, or NRG-1 protein, although other mammalian proteins can also be used, including rodents (mice, rats, hamsters, guinea pigs), farm animals (cows, sheep, pigs, horses) and primates. These latter methods can be used for the development of animal models of human disease. In some methods, variant or derivative ErbB receptor, dopamine receptor, glutamate receptor, or NRG-1 proteins can be used, including deletion ErbB receptor, dopamine receptor, glutamate receptor, or NRG-1 proteins as outlined above.

The assays herein utilize ErbB receptor, dopamine receptor, glutamate receptor, or NRG-1 proteins as defined herein. In some assays, portions of proteins can be utilized. In other assays, portions having different activities can be used. In addition, the assays described herein can utilize either isolated ErbB receptor, dopamine receptor, glutamate receptor, or NRG-1 proteins or cells comprising the ErbB receptor, dopamine receptor, glutamate receptor, or NRG-1 proteins. In some methods, the protein or the candidate agent is non-diffusably bound to an insoluble support having isolated sample receiving areas (e.g., a microtiter plate or an array). The insoluble supports can be made of any composition to which the compositions can be bound, is readily separated from soluble material, and is otherwise compatible with the overall method of screening. The surface of such supports can be solid or porous and of any convenient shape. Examples of suitable insoluble supports include microtiter plates, arrays, membranes and beads. These are typically made of glass, plastic (e.g., polystyrene), polysaccharides, nylon or nitrocellulose, and Teflon™. Microtiter plates and arrays are especially convenient because a large number of assays can be carried out simultaneously, using small amounts of reagents and samples. In some cases magnetic beads are included. The particular manner of binding of the composition is not crucial so long as it is compatible with the reagents and overall methods described herein, maintains the activity of the composition and is nondiffusable. Methods of binding include the use of antibodies (which do not sterically block either the ligand binding site or activation sequence when the protein is bound to the support), direct binding to ionic supports, chemical crosslinking, or by the synthesis of the protein or agent on the surface. Following binding of the protein or agent, excess unbound material is removed by washing. The sample receiving areas can then be blocked through incubation with bovine serum albumin (BSA), casein or other innocuous protein or other moiety. Also included in the methods and compositions herein are screening assays wherein solid supports are not used.

In other methods, the ErbB receptor, dopamine receptor, glutamate receptor, or NRG-1 protein is bound to the support, and a candidate bioactive agent is added to the assay. Alternatively, the candidate agent is bound to the support and the protein is added. Novel binding agents include specific antibodies, non-natural binding agents identified in screens of chemical libraries, and peptide analogs. Of particular interest are screening assays for agents that have a low toxicity for human cells. A wide variety of assays can be used for this purpose, including labeled in vitro protein-protein binding assays, electrophoretic mobility shift assays, immunoassays for protein binding, functional assays (such as phosphorylation assays).

The determination of the binding of the candidate bioactive agent to an ErbB receptor, dopamine receptor, glutamate receptor, or NRG-1 protein can be done in a number of ways. In some methods, the candidate bioactive agent is labeled, and binding determined directly. For example, this can be done by attaching all or a portion of an ErbB receptor, dopamine receptor, glutamate receptor, or NRG-1 protein to a solid support, adding a labeled candidate agent (for example a fluorescent label), washing off excess reagent, and determining whether the label is present on the solid support. Various blocking and washing steps can be utilized.

By “labeled” herein is meant that the compound is either directly or indirectly labeled with a label which provides a detectable signal, e.g., radioisotope, fluorescers, enzyme, antibodies, particles such as magnetic particles, chemiluminescers, or specific binding molecules. Specific binding molecules include pairs, such as biotin and streptavidin, digoxin and antidigoxin. For the specific binding members, the complementary member would normally be labeled with a molecule which provides for detection, in accordance with known procedures, as outlined above. The label can directly or indirectly provide a detectable signal.

In some methods, only one of the components is labeled. For example, the proteins (or proteinaceous candidate agents) can be labeled at tyrosine positions using ¹²⁵I, or with fluorophores. Alternatively, more than one component can be labeled with different labels; using ¹²⁵I for the proteins, for example, and a fluorophor for the candidate agents.

In other methods, the binding of the candidate bioactive agent is determined through the use of competitive binding assays. In this method, the competitor is a binding moiety known to bind to the target molecule such as an antibody, peptide, binding partner, or ligand. Under certain circumstances, there can be competitive binding as between the bioactive agent and the binding moiety, with the binding moiety displacing the bioactive agent. This assay can be used to determine candidate agents which interfere with binding between proteins and the competitor.

In some methods, the candidate bioactive agent is labeled. Either the candidate bioactive agent, or the competitor, or both, is added first to the protein for a time sufficient to allow binding, if present. Incubations can be performed at any temperature which facilitates optimal activity, typically between about 4° C. and 40° C. Incubation periods are selected for optimum activity, but can also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In other methods, the competitor is added first, followed by the candidate bioactive agent. Displacement of the competitor is an indication that the candidate bioactive agent is binding to the ErbB receptor, dopamine receptor, glutamate receptor, or NRG-1 protein and thus is capable of binding to, and potentially modulating, the activity of the protein. In this method, either component can be labeled. For example, if the competitor is labeled, the presence of label in the wash solution indicates displacement by the agent. Alternatively, if the candidate bioactive agent is labeled, the presence of the label on the support indicates displacement.

In other methods, the candidate bioactive agent is added first, with incubation and washing, followed by the competitor. The absence of binding by the competitor can indicate that the bioactive agent is bound to the ErbB receptor, dopamine receptor, glutamate receptor, or NRG-1 protein with a higher affinity. Thus, if the candidate bioactive agent is labeled, the presence of the label on the support, coupled with a lack of competitor binding, can indicate that the candidate agent is capable of binding to the protein.

Competitive binding methods can also be run as differential screens. These methods can comprise an ErbB receptor, dopamine receptor, glutamate receptor, or NRG-1 protein and a competitor in a first sample. A second sample comprises a candidate bioactive agent, an ErbB receptor, dopamine receptor, glutamate receptor, or NRG-1 protein and a competitor. The binding of the competitor is determined for both samples, and a change, or difference in binding between the two samples indicates the presence of an agent capable of binding to the ErbB receptor, dopamine receptor, glutamate receptor, or NRG-1 protein and potentially modulating its activity. If the binding of the competitor is different in the second sample relative to the first sample, the agent is capable of binding to the protein.

Other methods utilize differential screening to identify drug candidates that bind to the native ErbB receptor, dopamine receptor, glutamate receptor, or NRG-1 protein, but cannot bind to modified proteins. The structure of the protein can be modeled, and used in rational drug design to synthesize agents that interact with that site. Drug candidates that affect ErbB receptor signaling and/or dopaminergic transmission signaling, or glutamatergic transmission signaling bioactivity are also identified by screening drugs for the ability to either enhance or reduce the activity of the protein.

In some methods, screening for agents that modulate the activity of proteins are performed. In general, this will be done on the basis of the known biological activity of the ErbB receptor, dopamine receptor, glutamate receptor, or NRG-1 protein. In these methods, a candidate bioactive agent is added to a sample of the protein, as above, and an alteration in the biological activity of the protein is determined. “Modulating the activity” includes an increase in activity, a decrease in activity, or a change in the type or kind of activity present. Thus, in these methods, the candidate agent should both bind to ErbB receptor, dopamine receptor, glutamate receptor, or NRG-1 (although this may not be necessary), and alter its biological or biochemical activity as defined herein. The methods include both in vitro screening methods, as are generally outlined above, and in vivo screening of cells for alterations in the presence, distribution, activity or amount of the protein.

Some methods comprise combining an ErbB receptor, dopamine receptor, glutamate receptor, or NRG-1 sample and a candidate bioactive agent, then evaluating the effect on ErbB receptor, dopamine receptor, or glutamate receptor activity to treat symptoms of a psychiatric disorder. By “ErbB receptor, dopamine receptor, glutamate receptor activity” or grammatical equivalents herein is meant one of ErbB receptor, dopamine receptor, or glutamate receptor biological activities, including, but not limited to, its ability to affect immune activation or inhibition. One activity herein is the capability to bind to a target gene, or modulate ErbB receptor signaling and/or dopaminergic transmission signaling, or glutamatergic transmission signaling, wherein ErbB receptor signaling and/or dopaminergic transmission signaling, or glutamatergic transmission signaling is induced or maintained.

In some methods, the activity of the ErbB receptor, dopamine receptor, glutamate receptor, or NRG-1 protein is decreased. In other methods, the activity of the ErbB receptor, dopamine receptor, glutamate receptor, or NRG-1 protein is increased. Thus, bioactive agents that are antagonists are useful in some methods, and bioactive agents that are agonists are useful in other methods. Combinations of antagonists and agonist can be used to treat a psychiatric disorder.

Methods for screening for bioactive agents capable of modulating the activity of an ErbB receptor, dopamine receptor, glutamate receptor, or NRG-1 protein are provided. These methods comprise adding a candidate bioactive agent, as defined above, to a cell. Cell types include a variety of cell types, including neuronal cells and hippocampal neuronal cells. The cells contain a recombinant nucleic acid that encodes an ErbB receptor, dopamine receptor, glutamate receptor, or NRG-1 protein. In one method, a library of candidate agents are tested on a plurality of cells. The effect of the candidate agent on ErbB receptor signaling and/or dopaminergic transmission signaling, or glutamatergic transmission signaling activity is then evaluated.

Positive controls and negative controls can be used in the assays. All control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples can be counted in a scintillation counter to determine the amount of bound compound.

A variety of other reagents can be included in the screening assays. These include reagents like salts, neutral proteins (e.g., albumin and detergents) which can be used to facilitate optimal protein-protein binding and/or reduce non-specific or background interactions. Reagents that otherwise improve the efficiency of the assay, (such as protease inhibitors, nuclease inhibitors, anti-microbial agents) can also be used. The mixture of components can be added in any order that provides for the requisite binding.

The components provided herein for the assays provided herein can also be combined to form kits. The kits can be based on the use of the protein and/or the nucleic acid encoding the ErbB receptor, dopamine receptor, glutamate receptor, or NRG-1 proteins. Assays regarding the use of nucleic acids are further described below.

(B) Animal Models

In one method, nucleic acids which encode ErbB receptor, dopamine receptor, glutamate receptor, or NRG-1 proteins or their modified forms can also be used to generate either transgenic animals, including “knock-in” and “knock out” animals which, in turn, are useful in the development and screening of therapeutically useful reagents. A non-human transgenic animal (e.g., a mouse or rat) is an animal having cells that contain a transgene, which transgene is introduced into the animal or an ancestor of the animal at a prenatal, e.g., an embryonic stage. A transgene is a DNA which is integrated into the genome of a cell from which a transgenic animal develops, and can include both the addition of all or part of a gene or the deletion of all or part of a gene. In some methods, cDNA encoding an ErbB receptor, dopamine receptor, glutamate receptor, or NRG-1 protein can be used to clone genomic DNA encoding an ErbB receptor, dopamine receptor, glutamate receptor, or NRG-1 protein in accordance with established techniques and the genomic sequences used to generate transgenic animals that contain cells which either express (or overexpress) or suppress the desired DNA. Methods for generating transgenic animals, particularly animals such as mice or rats, have become conventional in the art and are described, for example, in U.S. Pat. Nos. 4,736,866 and 4,870,009, each incorporated herein by reference in their entirety. Typically, particular cells would be targeted for a ErbB receptor, dopamine receptor, glutamate receptor, or NRG-1 protein transgene incorporation with tissue-specific enhancers. Transgenic animals that include a copy of a transgene encoding a ErbB receptor, dopamine receptor, glutamate receptor, or NRG-1 protein introduced into the germ line of the animal at an embryonic stage can be used to examine the effect of increased expression of the desired nucleic acid. Such animals can be used as tester animals for reagents thought to confer protection from, for example, pathological conditions associated with its overexpression. In accordance with this facet, an animal is treated with the reagent and a reduced incidence of the pathological condition, compared to untreated animals bearing the transgene, would indicate a potential therapeutic intervention for the pathological condition. Similarly, non-human homologues of an ErbB receptor, dopamine receptor, glutamate receptor, or NRG-1 protein can be used to construct a transgenic animal comprising a protein “knock out” animal which has a defective or altered gene encoding an ErbB receptor, dopamine receptor, glutamate receptor, or NRG-1 protein as a result of homologous recombination between the endogenous gene encoding an ErbB receptor, dopamine receptor, glutamate receptor, or NRG-1 protein and altered genomic DNA encoding the protein introduced into an embryonic cell of the animal. For example, cDNA encoding an ErbB receptor, dopamine receptor, glutamate receptor, or NRG-1 protein can be used to clone genomic DNA encoding the protein in accordance with established techniques. A portion of the genomic DNA encoding an ErbB receptor, dopamine receptor, glutamate receptor, or NRG-1 protein can be deleted or replaced with another gene, such as a gene encoding a selectable marker which can be used to monitor integration. Typically, several kilobases of unaltered flanking DNA (both at the 5′ and 3′ ends) are included in the vector (see, e.g., Thomas and Capecchi, Cell 51:503, 1987, incorporated herein by reference in its entirety, for a description of homologous recombination vectors). The vector is introduced into an embryonic stem cell line (e.g., by electroporation) and cells in which the introduced DNA has homologously recombined with the endogenous DNA are selected (see, e.g., Li et al., Cell 69:915, 1992, incorporated herein by reference in its entirety). The selected cells are then injected into a blastocyst of an animal (e.g., a mouse or rat) to form aggregation chimeras (see, e.g., Bradley, in Teratocarcinomas and Embryonic Stem Cells: A Practical Approach, E. J. Robertson, ed. (IRL, Oxford, 1987), pp. 113-152). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of an ErbB receptor, dopamine receptor, glutamate receptor, or NRG-1 protein polypeptide.

Animal models for ErbB receptor signaling and/or dopaminergic transmission signaling, or glutamatergic transmission signaling related disorders, or having a particular state of ErbB receptor signaling and/or dopaminergic transmission signaling, or glutamatergic transmission signaling activity can include, for example, genetic models of psychiatric disorders. For example, such animal models for psychiatric disorders can include NRG-1 knockout mice (lines harboring mutations in either the EGF-like, Ig-like or CRD-specific domains), NRG-1 floxed conditional knockout mice (i.e., where exons encoding NRG-1 functional domains are flanked by recombination sequences for cre recombinase), NRG-2 and NRG-3 knockout mice, ErbB4^{-/-} HER4 rescued (i.e., ErbB4 knockouts rescued from embryonic lethality by expression of erbB4 in heart), ErbB2 floxed conditional mutant mice and dopamine D2R, D3R and D4R receptor mutant mice. Other models can include studies involving treatment of psychiatric disorders.

Animal models exhibiting ErbB receptor signaling and/or dopaminergic transmission signaling, or glutamatergic transmission signaling related disorder-like symptoms can be engineered by utilizing, for example, ErbB receptor, dopamine receptor, glutamate receptor, or NRG-1 sequences in conjunction with techniques for producing transgenic animals that are well known to those of skill in the art. For example, gene sequences can be introduced into, and overexpressed in, the genome of the animal of interest, or, if endogenous target gene sequences are present, they can either be overexpressed or, alternatively, can be disrupted in order to underexpress or inactivate target gene expression.

In order to overexpress a target gene sequence, the coding portion of the target gene sequence can be ligated to a regulatory sequence which is capable of driving gene expression in the animal and cell type of interest. Such regulatory regions will be well known to those of skill in the art, and can be utilized in the absence of undue experimentation.

For underexpression of an endogenous target gene sequence, such a sequence can be isolated and engineered such that when reintroduced into the genome of the animal of interest, the endogenous target gene alleles will be inactivated. The engineered target gene sequence is introduced via gene targeting such that the endogenous target sequence is disrupted upon integration of the engineered target sequence into the animal's genome.

Animals of any species, including, but not limited to, mice, rats, rabbits, guinea pigs, pigs, micro-pigs, goats, and non-human primates, e.g., baboons, monkeys, and chimpanzees can be used to generate animal models of ErbB receptor signaling and/or dopaminergic transmission signaling, or glutamatergic transmission signaling related disorders or being a perpetually desired state of the ErbB receptor signaling and/or dopaminergic transmission signaling, or glutamatergic transmission signaling.

(C) Nucleic Acid Based Therapeutics

Nucleic acids encoding ErbB receptor, dopamine receptor, glutamate receptor, or NRG-1 polypeptides, antagonists or agonists can also be used in gene therapy. Broadly speaking, a gene therapy vector is an exogenous polynucleotide which produces a medically useful phenotypic effect upon the mammalian cell(s) into which it is transferred. A vector can or can not have an origin of replication. For example, it is useful to include an origin of replication in a vector for propagation of the vector prior to administration to a patient. However, the origin of replication can often be removed before administration if the vector is designed to integrate into host chromosomal DNA or bind to host mRNA or DNA. Vectors used in gene therapy can be viral or nonviral. Viral vectors are usually introduced into a patient as components of a virus. Nonviral vectors, typically dsDNA, can be transferred as naked DNA or associated with a transfer-enhancing vehicle, such as a receptor-recognition protein, lipoamine, or cationic lipid.

Viral vectors, such as retroviruses, adenoviruses, adenoassociated viruses and herpes viruses, are often made up of two components, a modified viral genome and a coat structure surrounding it (see generally Smith et al., Ann. Rev. Microbiol. 49:807-838, 1995, incorporated herein by reference in its entirety), although sometimes viral vectors are introduced in naked form or coated with proteins other than viral proteins. Most current vectors have coat structures similar to a wildtype virus. This structure packages and protects the viral nucleic acid and provides the means to bind and enter target cells. However, the viral nucleic acid in a vector designed for gene therapy is changed in many ways. The goals of these changes are to disable growth of the virus in target cells while maintaining its ability to grow in vector form in available packaging or helper cells, to provide space within the viral genome for insertion of exogenous DNA sequences, and to incorporate new sequences that encode and enable appropriate expression of the gene of interest. Thus, vector nucleic acids generally comprise two components: essential cis-acting viral sequences for replication and packaging in a helper line and the transcription unit for the exogenous gene. Other viral functions are expressed in trans in a specific packaging or helper cell line.

Nonviral nucleic acid vectors used in gene therapy include plasmids, RNAs, antisense oligonucleotides (e.g., methylphosphonate or phosphorothiolate), polyamide nucleic acids, interfering RNA (RNAi), hairpin RNA, and yeast artificial chromosomes (YACs). Such vectors typically include an expression cassette for expressing a protein or RNA. The promoter in such an expression cassette can be constitutive, cell type-specific, stage-specific, and/or modulatable (e.g., by hormones such as glucocorticoids; MMTV promoter). Transcription can be increased by inserting an enhancer sequence into the vector. Enhancers are cis-acting sequences of between 10 to 300 bp that increase transcription by a promoter. Enhancers can effectively increase transcription when either 5′ or 3′ to the transcription unit. They are also effective if located within an intron or within the coding sequence itself. Typically, viral enhancers are used, including SV40 enhancers, cytomegalovirus enhancers, polyoma enhancers, and adenovirus enhancers. Enhancer sequences from mammalian systems are also commonly used, such as the mouse immunoglobulin heavy chain enhancer.

Gene therapy vectors can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Modulating Signaling in ErbB Receptor Signaling and/or Dopaminergic Transmission Signaling, or Glutamatergic Transmission Signaling Pathway

(A) Assays for Modulators of ErbB Receptor Signaling and/or Dopaminergic Transmission Signaling, or Glutamatergic Transmission Signaling

In numerous embodiments of this invention, the level of ErbB receptor signaling and/or dopaminergic transmission signaling, or glutamatergic transmission signaling will be modulated in a cell by administering to the cell, in vivo or in vitro, any of a large number of ErbB receptor, dopamine receptor, or glutamate receptor-modulating molecules, e.g., polypeptides, antibodies, amino acids, nucleotides, lipids, carbohydrates, or any organic or inorganic molecule.

To identify molecules capable of modulating ErbB receptor signaling and/or dopaminergic transmission signaling, or glutamatergic transmission signaling, assays will be performed to detect the effect of various compounds on ErbB receptor signaling and/or dopaminergic transmission signaling, or glutamatergic transmission signaling activity in a cell. ErbB receptor signaling and/or dopaminergic transmission signaling, or glutamatergic transmission signaling can be assessed using a variety of in vitro and in vivo assays to determine functional, chemical, and physical effects, e.g., measuring the binding of ErbB receptor, dopamine receptor, or glutamate receptor to other molecules (e.g., radioactive binding to NRG-1 or glutamate), measuring protein and/or RNA levels of ErbB receptor, dopamine receptor, or glutamate receptor signaling via NRG-1 or glutamate that provides an anti-psychotic drug response, or measuring other aspects of pathway signaling, e.g., phosphorylation levels, transcription levels, receptor activity, ligand binding. Such assays can be used to test for modulators, inhibitors, and activators of ErbB receptor signaling and/or dopaminergic transmission signaling, or glutamatergic transmission signaling. Modulators thus identified are useful for, e.g., many diagnostic and therapeutic applications.

The ErbB receptor, dopamine receptor, or glutamate receptor signaling via NRG-1 or glutamate in the assay will typically be a recombinant or naturally occurring polypeptide or a conservatively modified variant thereof. Alternatively, the ErbB receptor signaling and/or dopaminergic transmission signaling, e.g., dopamine D2 type receptors, or glutamatergic transmission signaling via NRG-1 or glutamate in the assay will be derived from a eukaryote and include an amino acid subsequence having amino acid sequence identity to the naturally occurring ErbB receptor signaling and/or dopaminergic transmission signaling, or glutamatergic transmission signaling. Generally, the amino acid sequence identity will be at least 70%, optionally at least 75%, 85%, or 86%, 87%, 88%, 89%, 90%, 91%, 92%, 93%, 94%, 95%, 96%, 97%, 98% or greater. Optionally, the polypeptide of the assays will comprise a domain of an ErbB receptor, dopamine receptor, glutamate receptor, or NRG-1. In certain embodiments, a domain of ErbB receptor, dopamine receptor, glutamate receptor, or NRG-1 protein is bound to a solid substrate and used, e.g., to isolate any molecules that can bind to and/or modulate their activity. In certain embodiments, a domain of a ErbB receptor, dopamine receptor, glutamate receptor, or NRG-1 polypeptide, e.g., an N-terminal domain, a C-terminal domain, is fused to a heterologous polypeptide, thereby forming a chimeric polypeptide. Such chimeric polypeptides are also useful, e.g., in assays to identify modulators of ErbB receptor signaling and/or dopaminergic transmission signaling, e.g., dopamine D2 type receptors, or glutamatergic transmission signaling via NRG-1 or glutamate.

The terms “identical” or percent “identity,” in the context of two or more nucleic acids or polypeptide sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence or amino acid sequence encoding a ErbB receptor, dopamine receptor, glutamate receptor, or NRG-1 protein as described herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” This term also refers to, or can be applied to, the compliment of a test sequence. The term also includes sequences that have deletions and/or additions, as well as those that have substitutions. As described below, the preferred algorithms can account for gaps. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482, 1981 by the homology alignment algorithm of Needleman & Wunsch, J. Mol. Biol. 48:443, 1970 by the search for similarity method of Pearson & Lipman, Proc. Nat'l. Acad. Sci. 85:2444, 1988 by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology, eds. 1995 supplement)).

A preferred example of algorithm that is suitable for determining percent sequence identity and sequence similarity are the BLAST and BLAST 2.0 algorithms, which are described in Altschul et al., Nuc. Acids Res 25:3389-3402, 1977 and Altschul et al., J. Mol. Biol. 215:403-410, 1990 respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. 89:10915, 1989) alignments (B) of 50, expectation (E) of 10, M=5, N=−4, and a comparison of both strands.

“Polypeptide”, “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions can also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., Ausubel et al, supra.

For PCR, a temperature of about 36° C. is typical for low stringency amplification, although annealing temperatures can vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. PCR Protocols, A Guide to Methods and Applications, Academic Press, Inc. N.Y. (1990).

Samples or assays that are treated with a potential ErbB receptor signaling and/or dopaminergic transmission signaling, or glutamatergic transmission signaling inhibitor are compared to control samples without the test compound, to examine the extent of modulation. Control samples (untreated with activators or inhibitors) are assigned a relative ErbB receptor, dopamine receptor, or glutamate receptor activity value of 100. Inhibition of ErbB receptor signaling and/or dopaminergic transmission signaling, or glutamatergic transmission signaling is achieved when the ErbB receptor signaling and/or dopaminergic transmission signaling, or glutamatergic transmission signaling activity value relative to the control is about 90%, optionally about 50%, optionally about 25-0%. Activation of a ErbB receptor signaling and/or dopaminergic transmission signaling, or glutamatergic transmission signaling is achieved when the ErbB receptor signaling and/or dopaminergic transmission signaling, or glutamatergic transmission signaling activity value relative to the control is about 110%, optionally about 150%, 200-500%, or about 1000-2000%.

The effects of the test compounds upon the function of the polypeptides can be measured by examining any of the parameters described above. Any suitable physiological change that affects ErbB receptor signaling and/or dopaminergic transmission signaling, or glutamatergic transmission signaling activity can be used to assess the influence of a test compound on the polypeptides of this invention. When the functional consequences are determined using intact cells or animals, one can also measure a variety of effects such as changes in cell growth or changes in cell-cell interactions.

Modulators of ErbB receptor signaling and/or dopaminergic transmission signaling, or glutamatergic transmission signaling that act by modulating gene expression can also be identified. For example, a host cell containing an ErbB receptor, dopamine receptor, glutamate receptor, or NRG-1 protein of interest is contacted with a test compound for a sufficient time to effect any interactions, and then the level of gene expression is measured. The amount of time to effect such interactions can be empirically determined, such as by running a time course and measuring the level of transcription as a function of time. The amount of transcription can be measured using any method known to those of skill in the art to be suitable. For example, mRNA expression of the protein of interest can be detected using Northern blots or by detecting their polypeptide products using immunoassays.

(B) Assays for Compounds that Modulate ErbB Receptor Signaling and/or Dopaminergic Transmission Signaling, or Glutamatergic Transmission Signaling

In certain embodiments, assays will be performed to identify molecules that physically interact with ErbB receptor, dopamine receptor, glutamate receptor, or NRG-1. Such molecules can be any type of molecule, including polypeptides, polynucleotides, amino acids, nucleotides, carbohydrates, lipids, or any other organic or inorganic molecule. Such molecules can represent molecules that normally interact with or ErbB receptor, dopamine receptor, glutamate receptor, or NRG-1 or can be synthetic or other molecules that are capable of interacting with ErbB receptor, dopamine receptor, glutamate receptor, or NRG-1 and that can potentially be used as lead compounds to identify classes of molecules that can interact with and/or modulate ErbB receptor signaling and/or dopaminergic transmission signaling, e.g., dopamine D2 type receptors, or glutamatergic transmission signaling via NRG-1 or glutamate ErbB receptor signaling and/or dopaminergic transmission signaling, e.g., dopamine D2 type receptors, or glutamatergic transmission signaling via NRG-1 or glutamate. Such assays can represent physical binding assays, such as affinity chromatography, immunoprecipitation, two-hybrid screens, or other binding assays, or can represent genetic assays.

In any of the binding or functional assays described herein, in vivo or in vitro, ErbB receptor signaling and/or dopaminergic transmission signaling, e.g., dopamine D2 type receptors, or glutamatergic transmission signaling via NRG-1 or glutamate, or any derivative, variation, homolog, or fragment of ErbB receptor, dopamine receptor, glutamate receptor, or NRG-1 can be used. Preferably, the ErbB receptor, dopamine receptor, glutamate receptor, or NRG-1 has at least about 85% identity to the amino acid sequence of the naturally occurring ErbB receptor, dopamine receptor, glutamate receptor, or NRG-1. In numerous embodiments, a fragment of a ErbB receptor, dopamine receptor, glutamate receptor, or NRG-1 is used. Such fragments can be used alone, in combination with other ErbB receptor, dopamine receptor, glutamate receptor, or NRG-1 protein fragments, or in combination with sequences from heterologous proteins, e.g., the fragments can be fused to a heterologous polypeptides, thereby forming a chimeric polypeptide.

Compounds that interact with ErbB receptor signaling and/or dopaminergic transmission signaling, e.g., dopamine D2 type receptors, or glutamatergic transmission signaling via NRG-1 or glutamate can be isolated based on an ability to specifically bind to a ErbB receptor, dopamine receptor, glutamate receptor, or NRG-1 protein or fragment thereof. In numerous embodiments, the ErbB receptor, dopamine receptor, glutamate receptor, NRG-1 protein or protein fragment will be attached to a solid support. In one embodiment, affinity columns are made using the ErbB receptor, dopamine receptor, glutamate receptor, or NRG-1 polypeptide, and physically-interacting molecules are identified. It will be apparent to one of skill that chromatographic techniques can be performed at any scale and using equipment from many different manufactures (e.g., Pharmacia Biotechnology). In addition, molecules that interact with ErbB receptor, dopamine receptor, glutamate receptor, or NRG-1 in vivo can be identified by co-immunoprecipitation or other methods, i.e., immunoprecipitating ErbB receptor, dopamine receptor, glutamate receptor, or NRG-1 using anti-NRG-1 or anti-ErbB receptor, anti-dopamine receptor, or anti-glutamate receptor antibodies from a cell or cell extract, and identifying compounds, e.g., proteins, that are precipitated along with the ErbB receptor, dopamine receptor, glutamate receptor, or NRG-1. Such methods are well known to those of skill in the art and are taught, e.g., in Ausubel et al., 1994; Sambrook et al., Molecular Cloning: A Laboratory Manual, Cold Spring Harbor Press, N.Y., 1989; and Harlow and Lane, Antibodies: A Laboratory Manual, Cold Spring Harbor Press, N.Y., 1989.

(C) Reducing ErbB Receptor, Dopamine Receptor, Glutamate Receptor, or NRG-1 Protein Activity Levels in Cells

In certain embodiments, this invention provides methods of treating a psychiatric disorder, e.g., schizophrenia or attention deficit disorder by reducing ErbB receptor signaling and/or dopaminergic transmission signaling, or glutamatergic transmission signaling or protein levels in a cell. Typically, such methods are used to reduce an elevated level of ErbB receptor, dopamine receptor, glutamate receptor, or NRG-1 protein, e.g., an elevated level in hippocampal slices or hippocampal neurons can be performed in any of a number of ways, e.g., lowering the copy number of ErbB receptor, dopamine receptor, glutamate receptor, or NRG-1 protein genes or decreasing the level of mRNA, protein, or protein activity in a cell. Preferably, the level of ErbB receptor, dopamine receptor, glutamate receptor, or NRG-1 protein activity is lowered to a level typical of normal hippocampal neurons, but the level can be reduced to any level that is sufficient to decrease ErbB receptor signaling and/or dopaminergic transmission signaling, or glutamatergic transmission signaling of the cell, including to levels above or below those typical of normal cells. Preferably, such methods involve the use of inhibitors of ErbB receptor, dopamine receptor, glutamate receptor, or NRG-1 protein, where an “inhibitor of ErbB receptor, dopamine receptor, glutamate receptor, or NRG-1 protein” is a molecule that acts to reduce ErbB receptor, dopamine receptor, glutamate receptor, or NRG-1 protein polynucleotide levels, polypeptide levels and/or protein activity. Such inhibitor s include, but are not limited to, small molecule inhibitors, antisense polynucleotides, ribozymes, antibodies, dominant negative ErbB receptor, dopamine receptor, glutamate receptor, or NRG-1 protein forms, and small molecule inhibitors of ErbB receptor, dopamine receptor, glutamate receptor, or NRG-1 protein.

In preferred embodiments, NRG-1 protein or glutamate levels or signaling through ErbB receptor, dopamine receptor, or glutamate receptor will be reduced so as to treat a psychiatric disorder as a result of inhibition of ErbB receptor signaling and/or dopaminergic transmission signaling, e.g., dopamine D2 type receptors, or glutamatergic transmission signaling via NRG-1 or glutamate. The proliferation of a cell refers to the rate at which the cell or population of cells divides, or to the extent to which the cell or population of cells divides or increases in number. Proliferation can reflect any of a number of factors, including the rate of cell growth and division and the rate of cell death. Without being bound by the following offered theory, it is suggested that compounds leading to modulation, e.g., inhibition or activation, of signaling via one or more of the ErbB receptor, dopamine receptor, glutamate receptor, or NRG-1 in hippocampal slices or brain hippocampal neurons is useful to treat a psychiatric disorder, e.g., schizophrenia or attention deficit disorder. Inhibition of signaling activity via ErbB receptor, dopamine receptor, glutamate receptor, or NRG-1 protein can act to treat psychiatric disorders, such as schizophrenia, bipolar disorder, autism and attention deficit disorders. The ability of any of the present compounds to affect ErbB receptor, dopamine receptor, glutamate receptor, or NRG-1 protein activity can be determined based on any of a number of factors, including, but not limited to, a polynucleotide level, e.g., mRNA or gDNA, the level of ErbB receptor, dopamine receptor, glutamate receptor, or NRG-1 polypeptide, the degree of binding of a compound to a ErbB receptor, dopamine receptor, glutamate receptor, or NRG-1 polynucleotide or polypeptide, ErbB receptor, dopamine receptor, glutamate receptor, or NRG-1 protein intracellular localization, or any functional properties of ErbB receptor, dopamine receptor, glutamate receptor, or NRG-1 protein, such as the ability of ErbB receptor, dopamine receptor, glutamate receptor, or NRG-1 protein activity to to treat a psychiatric disorder, e.g., schizophrenia or attention deficit disorder.

(D) Inhibitors of ErbB Receptor, Dopamine Receptor, Glutamate Receptor, or NRG-1 Polynucleotides

In certain embodiments, ErbB receptor, dopamine receptor, glutamate receptor, or NRG-1 protein activity is downregulated, or entirely inhibited, by the use of siRNA or antisense polynucleotide, i.e., a nucleic acid complementary to, and which can preferably hybridize specifically to, a coding mRNA nucleic acid sequence, e.g., ErbB receptor, dopamine receptor, glutamate receptor, or NRG-1 induced mRNA, or a subsequence thereof. Binding of the antisense polynucleotide to the mRNA reduces the translation and/or stability of the ErbB receptor, dopamine receptor, glutamate receptor, or NRG-1 induced mRNA.

In the context of this invention, antisense polynucleotides can comprise naturally-occurring nucleotides, or synthetic species formed from naturally-occurring subunits or their close homologs. Antisense polynucleotides can also have altered sugar moieties or inter-sugar linkages. Exemplary among these are the phosphorothioate and other sulfur containing species which are known for use in the art. All such analogs are comprehended by this invention so long as they function effectively to hybridize with ErbB receptor, dopamine receptor, glutamate receptor, or NRG-1 induced mRNA.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

In addition to antisense polynucleotides, ribozymes can be used to target and inhibit transcription of ErbB receptor, dopamine receptor, glutamate receptor, or NRG-1 protein. A ribozyme is an RNA molecule that catalytically cleaves other RNA molecules. Different kinds of ribozymes have been described, including group I ribozymes, hammerhead ribozymes, hairpin ribozymes, RNAse P, and axhead ribozymes (see, e.g., Castanotto et al., Adv. in Pharmacology 25: 289-317, 1994 for a general review of the properties of different ribozymes).

The general features of hairpin ribozymes are described, e.g., in Hampel et al., Nucl. Acids Res., 18: 299-304, 1990; Hampel et al., European Patent Publication No. 0 360 257, 1990; U.S. Pat. No. 5,254,678. Methods of preparing are well known to those of skill in the art (see, e.g., Wong-Staal et al., WO 94/26877; Ojwang et al., Proc. Natl. Acad. Sci. USA, 90: 6340-6344, 1993; Yamada et al., Human Gene Therapy 1: 39-45, 1994; Leavitt et al., Proc. Natl. Acad. Sci. USA, 92: 699-703, 1995; Leavitt et al., Human Gene Therapy 5: 1151-120, 1994; and Yamada et al., Virology 205: 121-126, 1994).

ErbB receptor, dopamine receptor, glutamate receptor, or NRG-1 protein activity can also be decreased by the addition of an inhibitor of the ErbB receptor, dopamine receptor, glutamate receptor, or NRG-1 protein. This can be accomplished in any of a number of ways, including by providing a dominant negative ErbB receptor, dopamine receptor, glutamate receptor, or NRG-1 polypeptide, e.g., a form of ErbB receptor, dopamine receptor, glutamate receptor, or NRG-1 protein that itself has no activity and which, when present in the same cell as a functional ErbB receptor, dopamine receptor, glutamate receptor, or NRG-1 protein, reduces or eliminates the ErbB receptor, dopamine receptor, glutamate receptor, or NRG-1 protein activity of the functional ErbB receptor, dopamine receptor, glutamate receptor, or NRG-1 protein. Design of dominant negative forms is well known to those of skill and is described, e.g., in Herskowitz, Nature 329:219-22, 1987. Also, inactive polypeptide variants (muteins) can be used, e.g., by screening for the ability to inhibit ErbB receptor, dopamine receptor, glutamate receptor, or NRG-1 protein activity. Methods of making muteins are well known to those of skill (see, e.g., U.S. Pat. Nos. 5,486,463; 5,422,260; 5,116,943; 4,752,585; and 4,518,504). In addition, any small molecule, e.g., any peptide, amino acid, nucleotide, lipid, carbohydrate, or any other organic or inorganic molecule can be screened for the ability to bind to or inhibit ErbB receptor, dopamine receptor, glutamate receptor, or NRG-1 protein activity, as described below.

(E) Increasing ErbB Receptor, Dopamine Receptor, Glutamate Receptor, or NRG-1 Protein Activity Levels in Cells

In certain embodiments, this invention provides methods of treating neoplastic disease, allogeneic tissue rejection, or graft vs. host disease by increasing ErbB receptor, dopamine receptor, glutamate receptor, or NRG-1 signaling or protein levels in a cell. Typically, such methods are used to increase a reduced level of ErbB receptor, dopamine receptor, glutamate receptor, or NRG-1 protein, e.g., a reduced level in hippocampal slices or hippocampal neurons, and can be performed in any of a number of ways, e.g., increasing the copy number of ErbB receptor, dopamine receptor, glutamate receptor, or NRG-1 genes or increasing the level of ErbB receptor, dopamine receptor, glutamate receptor, or NRG-1 mRNA, protein, or protein activity in a cell. Preferably, the level of protein activity is increased to a level typical of a normal, cell, but the level can be increased to any level that is sufficient to increase ErbB receptor, dopamine receptor, or glutamate receptor signaling in the hippocampal slices or hippocampal neurons, including to levels above or below those typical of normal cells. Preferably, such methods involve the use of activators of ErbB receptor, dopamine receptor, glutamate receptor, or NRG-1 protein, where an “activator of ErbB receptor, dopamine receptor, glutamate receptor, or NRG-1 protein” is a molecule that acts to increase ErbB receptor, dopamine receptor, glutamate receptor, or NRG-1 gene polynucleotide levels, polypeptide levels and/or protein activity. Such activators can include, but are not limited to, small molecule activators of ErbB receptor, dopamine receptor, glutamate receptor, or NRG-1 protein.

(F) Modulators and Binding Compounds

The compounds tested as modulators of a ErbB receptor, dopamine receptor, glutamate receptor, or NRG-1 protein can be any small chemical compound, or a biological entity, such as a protein, sugar, nucleic acid or lipid. Typically, test compounds will be small chemical molecules and peptides. Essentially any chemical compound can be used as a potential modulator or binding compound in the assays of the invention, although most often compounds can be dissolved in aqueous or organic (especially DMSO-based) solutions. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland).

In one preferred embodiment, high throughput screening methods involve providing a combinatorial chemical or peptide library containing a large number of potential therapeutic compounds (potential modulator or binding compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res. 37:487-493, 1991; and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913, 1993), vinylogous polypeptides (Hagihara et al., J. Amer. Chem. Soc. 114:6568, 1992), nonpeptidal peptidomimetics with glucose scaffolding (Hirschmann et al., J. Amer. Chem. Soc. 114:9217-9218, 1992), analogous organic syntheses of small compound libraries (Chen et al., J. Amer. Chem. Soc. 116:2661, 1994), oligocarbamates (Cho et al., Science 261:1303, 1993), and/or peptidyl phosphonates (Campbell et al., J. Org. Chem. 59:658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology 14:309-314, 1996; and PCT/US96/10287), carbohydrate libraries (see, e.g., Liang et al., Science, 274:1520-1522, 1996; and U.S. Pat. No. 5,593,853), small organic molecule libraries (see, e.g., benzodiazepines, Baum, C&EN, page 33, Jan. 18, 1993; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences, Columbia, Md.).

(G) Solid State and Soluble High Throughput Assays

In one embodiment, the invention provides soluble assays using molecules such as an N-terminal or C-terminal domain either alone or covalently linked to a heterologous protein to create a chimeric molecule. In another embodiment, the invention provides solid phase based in vitro assays in a high throughput format, where a domain, chimeric molecule, ErbB receptor, dopamine receptor, glutamate receptor, or NRG-1 protein, or cell or tissue expressing an ErbB receptor, dopamine receptor, glutamate receptor, or NRG-1 protein is attached to a solid phase substrate.

In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds is possible using the integrated systems of the invention. More recently, microfluidic approaches to reagent manipulation have been developed.

The molecule of interest can be bound to the solid state component, directly or indirectly, via covalent or non covalent linkage, e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers can also serve as tags, and include polypeptide sequences, such as poly-gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154, 1993 (describing solid phase synthesis of, e.g., peptides); Geysen et al., J. Immun. Meth. 102:259-274, 1987 (describing synthesis of solid phase components on pins); Frank & Doring, Tetrahedron 44:6031-6040, 1988 (describing synthesis of various peptide sequences on cellulose disks); Fodor et al., Science 251: 767-777, 1991; Sheldon et al., Clinical Chemistry 39:718-719, 1993; and Kozal et al., Nature Medicine 2:753-759, 1996 (all describing arrays of biopolymers fixed to solid substrates). Nonchemical approaches for fixing tag binders to substrates include other common methods, such as heat, cross-linking by UV radiation.

(H) Rational Drug Design Assays

Yet another assay for compounds that modulate ErbB receptor, dopamine receptor, glutamate receptor, or NRG-1 protein activity involves computer assisted drug design, in which a computer system is used to generate a three-dimensional structure of an ErbB receptor, dopamine receptor, glutamate receptor, or NRG-1 protein based on the structural information encoded by its amino acid sequence. The input amino acid sequence interacts directly and actively with a pre-established algorithm in a computer program to yield secondary, tertiary, and quaternary structural models of the protein. The models of the protein structure are then examined to identify regions of the structure that have the ability to bind. These regions are then used to identify compounds that bind to the protein.

The three-dimensional structural model of the protein is generated by entering protein amino acid sequences of at least 10 amino acid residues or corresponding nucleic acid sequences encoding an ErbB receptor, dopamine receptor, glutamate receptor, or NRG-1 polypeptide into the computer system. The nucleotide sequence encoding the polypeptide, or the amino acid sequence thereof, and conservatively modified versions thereof, of the naturally occurring gene sequence. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential modulator binding regions are identified by the computer system. Three-dimensional structures for potential modulators are generated by entering amino acid or nucleotide sequences or chemical formulas of compounds, as described above. The three-dimensional structure of the potential modulator is then compared to that of the ErbB receptor, dopamine receptor, glutamate receptor, or NRG-1 protein to identify compounds that bind to the protein. Binding affinity between the protein and compound is determined using energy terms to determine which compounds have an enhanced probability of binding to the protein.

Computer systems are also used to screen for mutations, polymorphic variants, alleles and interspecies homologs of ErbB receptor, dopamine receptor, glutamate receptor, or NRG-1 induced genes. Such mutations can be associated with disease states or genetic traits. GeneChip™ and related technology can also be used to screen for mutations, polymorphic variants, alleles and interspecies homologs. Once the variants are identified, diagnostic assays can be used to identify patients having such mutated genes. Identification of the mutated ErbB receptor, dopamine receptor, glutamate receptor, or NRG-1 induced genes involves receiving input of a first nucleic acid or amino acid sequence of the naturally occurring ErbB receptor, dopamine receptor, glutamate receptor, or NRG-1 induced gene, respectively, and conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in various ErbB receptor, dopamine receptor, glutamate receptor, or NRG-1 induced genes, and mutations associated with disease states and genetic traits.

### Diagnostic Methods

In addition to assays, the creation of animal models, and nucleic acid based therepeutics, identification of important genes allows the use of these genes in diagnosis (e.g., diagnosis of cell states and abnormal cell conditions). Disorders based on mutant or variant ErbB receptor, dopamine receptor, glutamate receptor, or NRG-1 genes can be determined. Methods for identifying cells containing variant ErbB receptor, dopamine receptor, glutamate receptor, or NRG-1 genes comprising determining all or part of the sequence of at least one endogeneous genes in a cell are provided. As will be appreciated by those in the art, this can be done using any number of sequencing techniques. Methods of identifying the genotype of an individual comprising determining all or part of the sequence of at least one ErbB receptor, dopamine receptor, glutamate receptor, or NRG-1 gene of the individual are also provided. This is generally done in at least one tissue of the individual, and can include the evaluation of a number of tissues or different samples of the same tissue. The method can include comparing the sequence of the sequenced mutant ErbB receptor, dopamine receptor, glutamate receptor, or NRG-1 gene to a known ErbB receptor, dopamine receptor, glutamate receptor, or NRG-1 gene, i.e., a wild-type gene.

The sequence of all or part of the ErbB receptor, dopamine receptor, glutamate receptor, or NRG-1 gene can then be compared to the sequence of a known ErbB receptor, dopamine receptor, glutamate receptor, or NRG-1 gene to determine if any differences exist. This can be done using any number of known sequence identity programs, such as Bestfit, and others outlined herein. In some methods, the presence of a difference in the sequence between the ErbB receptor, dopamine receptor, glutamate receptor, or NRG-1 gene of the patient and the known ErbB receptor, dopamine receptor, glutamate receptor, or NRG-1 gene is indicative of a disease state or a propensity for a disease state, as outlined herein.

Similarly, diagnosis of psychiatric disorder as measured in hippocampal slices or hippocampal neuron cell states can be performed using the methods and compositions herein. By evaluating the gene expression profile of hippocampal neurons from a patient, the hippocampal neurons cell state can be determined. This is particularly useful to verify the action of a drug, for example an antipsychotic drug. Other methods comprise administering the drug to a patient and removing a biopsy cell sample, particularly of hippocampal neurons, from the patient. The gene expression profile of the cell is then evaluated, as outlined herein, for example by comparing it to the expression profile from an equivalent sample from a healthy individual. In this manner, both the efficacy (i.e., whether the correct expression profile is being generated from the drug) and the dose (is the dosage correct to result in the correct expression profile) can be verified.

The present discovery relating to the interacting role of ErbB receptor, dopamine receptor, glutamate receptor, or NRG-1 in treating a psychiatric disorder, e.g., schizophrenia or attention deficit disorder, thus provides methods for treating differing disease states. In one method, the ErbB receptor, dopamine receptor, glutamate receptor, or NRG-1 proteins, and particularly ErbB receptor, dopamine receptor, glutamate receptor, or NRG-1 protein fragments, are useful in the study or treatment of conditions which are mediated by various disease states, i.e., to diagnose, treat or prevent psychiatric disorders. Thus, “psychiatric disorder” can include conditions involving, for example, treat conditions including but not limited to, schizophrenia, bipolar depression, autism, or attention deficit disorder.

Methods for diagnosing a condition related to a psychiatric disorder is provided in which brain hippocampal neuron activity in an individual is measured. The methods comprise measuring the activity of ErbB receptor, dopamine receptor, glutamate receptor, or NRG-1 protein in a tissue from the individual or patient, which can include a measurement of the amount or specific activity of the protein. This activity is compared to the activity of ErbB receptor, dopamine receptor, glutamate receptor, or NRG-1 protein from either an unaffected second individual or from an unaffected tissue from the first individual. When these activities are different, the first individual can be at risk for a psychiatric disorder as measured by hippocampal slice or hippocampal neuron cell activity.

Furthermore, nucleotide sequences encoding a ErbB receptor, dopamine receptor, glutamate receptor, or NRG-1 protein can also be used to construct hybridization probes for mapping the gene which encodes that ErbB receptor, dopamine receptor, glutamate receptor, or NRG-1 protein and for the genetic analysis of individuals with genetic disorders. The nucleotide sequences provided herein can be mapped to a chromosome and specific regions of a chromosome using known techniques, such as in situ hybridization, linkage analysis against known chromosomal markers, and hybridization screening with libraries.

### Peptides and Polypeptides

The invention provides isolated or recombinant polypeptides comprising an amino acid sequence having at least 95%, 96%, 97%, 98%, 99% or more sequence identity to ErbB receptor, dopamine receptor, glutamate receptor, or NRG-1 polypeptides over a region of at least about 100, 150, 200, 250, 300, 350, 400, 450, 500, 550, 600, 650, 700, 750, 800, 850, 900, 950, 1000, 1100 or more residues, or, the full length of the polypeptide, or, a polypeptide encoded by a nucleic acid of the invention. The invention provides methods for inhibiting the activity of ErbB receptor, dopamine receptor, glutamate receptor, or NRG-1 polypeptide, e.g., a polypeptide of the invention. The invention also provides methods for screening for compositions that inhibit the activity of, or bind to (e.g., bind to the active site), of ErbB receptor, dopamine receptor, glutamate receptor, or NRG-1 polypeptides, e.g., a polypeptide of the invention.

In one aspect, the invention provides ErbB receptor, dopamine receptor, glutamate receptor, or NRG-1 polypeptides (and the nucleic acids encoding them) where one, some or all of the ErbB receptor, dopamine receptor, glutamate receptor, or NRG-1 polypeptides replacement with substituted amino acids. In one aspect, the invention provides methods to disrupt the interaction of ErbB receptor, dopamine receptor, glutamate receptor, or NRG-1 polypeptides with other proteins, in pathways related to a psychiatric disorder, e.g., schizophrenia or attention deficit disorder.

The peptides and polypeptides of the invention can be expressed recombinantly in vivo after administration of nucleic acids, as described above, or, they can be administered directly, e.g., as a pharmaceutical composition. They can be expressed in vitro or in vivo to screen for modulators of a ErbB receptor, dopamine receptor, glutamate receptor, or NRG-1 activity and for agents that can treat or ameliorate a psychiatric disorder, e.g., schizophrenia or attention deficit disorder.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, A. K., Therapeutic Peptides and Proteins, Formulation, Processing and Delivery Systems Technomic Publishing Co., Lancaster, Pa., 1995. For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 431A Peptide Synthesizer (Perkin Elmer) in accordance with the instructions provided by the manufacturer.

The peptides and polypeptides of the invention, as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, it has an ErbB receptor, dopamine receptor, glutamate receptor, or NRG-1 activity.

In one aspect, the polypeptide or peptidomimetic composition can be a dominant-negative mutant within the scope of the invention if it can inhibit an activity of ErbB receptor, dopamine receptor, glutamate receptor, of the invention, e.g., be a dominant-negative mutant or bind to NRG-1 polypeptide or other ligand of the invention. The dominant negative mutant can be a peptide or peptide mimetic that can inhibit an activity of a ErbB receptor, dopamine receptor, glutamate receptor, or NRG-1, or a nucleic acid composition, in the form of a DNA vector or gene therapy vector, that expresses a dominant-negative polypeptide that can inhibit an activity of ErbB receptor, dopamine receptor, glutamate receptor, or NRG-1. The dominant negative mutant can bind to a ligand of the kinase or a target target, affecting ligand target interaction. The dominant negative molecule can act, for example, by blocking protein protein interactions, or by blocking phosphorylation of the kinase. An example of a dominant negative peptide is a peptide with a mutation in a lysine residue in the ATP binding domain of the ErbB receptor, dopamine receptor, or glutamate receptor, as described herein, that inhibits ErbB receptor, dopamine receptor, or glutamate receptor activity. A further example of a dominant negative peptide is a peptide with a mutation in the SH2 domain or SH3 domain of the ErbB receptor, dopamine receptor, glutamate receptor, or NRG-1 as described herein, that inhibits ErbB receptor, dopamine receptor, glutamate receptor, or NRG-1 activity.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, N.Y.).

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from ErbB receptor, dopamine receptor, glutamate receptor, or NRG-1 polypeptide of the invention, resulting in modification of the structure of the polypeptide, without significantly altering its biological activity. For example, amino- or carboxyl-terminal, or internal, amino acids which are not required for ErbB receptor, dopamine receptor, glutamate receptor, or NRG-1 activity or interaction can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., N.Y. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9: 205-223, 1998; Hruby, Curr. Opin. Chem. Biol. 1: 114-119, 1997; Ostergaard, Mol. Divers. 3: 17-27, 1997; Ostresh, Methods Enzymol. 267: 220-234, 1996. Modified peptides of the invention can be further produced by chemical modification methods, see, e.g., Belousov, Nucleic Acids Res. 25: 3440-3444, 1997; Frenkel, Free Radic. Biol. Med. 19: 373-380, 1995; Blommers, Biochemistry 33: 7886-7896, 1994.

Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Amgen Inc., Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams, Biochemistry 34: 1787-1797, 1995; Dobeli, Protein Expr. Purif. 12: 404-14, 1998). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll, DNA Cell. Biol., 12: 441-53, 1993.

### Treatment Regimes

The invention provides pharmaceutical compositions comprising one or a combination of small molecule chemical inhibitors, siRNA inhibitors, or dominant-negative mutants of ErbB receptor tyrosine kinase, dopamine receptor, glutamate receptor, or NRG-1 activity, (monoclonal, polyclonal or single chain Fv, intact or binding fragments thereof) or nucleic acid compositions, e.g., antisense oligonucleotides, double stranded RNA oligonucleotides (RNAi) or DNA oligonucleotides (vectors) containing nucleotide sequences encoding for the transcription of shRNA molecules, formulated together with a pharmaceutically acceptable carrier. Some compositions include a combination of multiple (e.g., two or more) small chemical molecules, siRNA molecules, monoclonal antibodies or antigen-binding portions thereof of the invention. In some compositions, each of the antibodies or antigen-binding portions thereof of the composition is a monoclonal antibody or a human sequence antibody that binds to a distinct, pre-selected epitope of an antigen.

In prophylactic applications, pharmaceutical compositions or medicaments are administered to a patient susceptible to, or otherwise at risk of a disease or condition (i.e., an immune disease) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient therapeutic response to the psychiatric disorder has been achieved. Typically, the therapeutic response is monitored and repeated dosages are given if the response starts to wane.

### Effective Dosages

Effective doses of the small molecule chemical inhibitors, siRNA inhibitors, or dominant-negative mutants of ErbB receptor tyrosine kinase, dopamine receptor, glutamate receptor, or NRG-1 activity, or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of psychiatric disorders described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration with a small chemical molecule, nucleic acid, siRNA, or antibody composition, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more small chemical molecules or siRNA molecules with different binding specificities are administered simultaneously, in which case the dosage of each small chemical molecule, siRNA molecule, or antibody administered falls within the ranges indicated. Small chemical molecule, siRNA molecule, or antibody is usually administered on multiple occasions. Intervals between single dosages can be weekly, monthly or yearly. Intervals can also be irregular as indicated by measuring blood levels of small chemical molecule, siRNA molecule, or antibody in the patient. In some methods, dosage is adjusted to achieve an antibody concentration of 1-1000 μg/ml and in some methods 25-300 μg/ml. Alternatively, antibody can be administered as a sustained release formulation, in which case less frequent administration is required. Dosage and frequency vary depending on the half-life of the compound in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Doses for small chemical molecules, siRNA molecules, or nucleic acids range from about 10 ng to 1 g, 100 ng to 100 mg, 1 μg to 10 mg, or 30-300 μg DNA per patient. Doses for infectious viral vectors vary from 10-100, or more, virions per dose.

### Prodrugs

The present invention is also related to prodrugs of the agents obtained by the methods disclosed herein. Prodrugs are agents which are converted in vivo to active forms (see, e.g., R. B. Silverman, 1992, The Organic Chemistry of Drug Design and Drug Action, Academic Press, Chp. 8). Prodrugs can be used to alter the biodistribution (e.g., to allow agents which would not typically enter the reactive site of the protease) or the pharmacokinetics for a particular agent. For example, a carboxylic acid group, can be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate agent which subsequently decomposes to yield the active agent. The prodrug moieties can be metabolized in vivo by esterases or by other mechanisms to carboxylic acids.

Examples of prodrugs and their uses are well known in the art (see, e.g., Berge et al., “Pharmaceutical Salts”, J. Pharm. Sci. 66: 1-19, 1977). The prodrugs can be prepared in situ during the final isolation and purification of the agents, or by separately reacting the purified agent in its free acid form with a suitable derivatizing agent. Carboxylic acids can be converted into esters via treatment with an alcohol in the presence of a catalyst.

Examples of cleavable carboxylic acid prodrug moieties include substituted and unsubstituted, branched or unbranched lower alkyl ester moieties, (e.g., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, dilower alkyl amides, and hydroxy amides.

### Routes of Administration

Methods of treatment of psychiatric disorders comprise administering modulators of ErbB inhibitors that can cross the blood brain barrier, for example, small chemical compounds. Small chemical molecule, siRNA molecule, or antibody compositions for treatment or amelioration of psychiatric disorder, or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of psychiatric disorder, can be administered by parenteral, topical, intravenous, oral, subcutaneous, intraarterial, intracranial, intraperitoneal, intranasal or intramuscular means for prophylactic as inhalants for small chemical molecule, siRNA molecule or antibody preparations targeting psychiatric disorder, and/or therapeutic treatment. The most typical route of administration of an immunogenic agent is subcutaneous although other routes can be equally effective. The next most common route is intramuscular injection. This type of injection is most typically performed in the arm or leg muscles. In some methods, agents are injected directly into a particular tissue where a tumor is found, for example intracranial injection or convection enhanced delivery. Intramuscular injection or intravenous infusion are preferred for administration of antibody. In some methods, particular therapeutic antibodies are delivered directly into the cranium. In some methods, antibodies are administered as a sustained release composition or device, such as a Medipad™ device.

Agents of the invention can optionally be administered in combination with other agents that are at least partly effective in treating various psychiatric diseases including schizophrenia and attention deficit disorder. In the case of infection in the brain, agents of the invention can also be administered in conjunction with other agents that increase passage of the agents of the invention across the blood-brain barrier (BBB).

### Administration Across the Blood-Brain Barrier

Nitric oxide is a vasodilator of the peripheral vasculature in normal tissue of the body. Increasing generation of nitric oxide by nitric oxide synthase causes vasodilation without loss of blood pressure. The blood-pressure-independent increase in blood flow through brain tissue increases cerebral bioavailability of blood-born compositions. This increase in nitric oxide can be stimulated by administering L-arginine. As nitric oxide is increased, cerebral blood flow is consequently increased, and drugs in the blood stream are carried along with the increased flow into brain tissue. Therefore, L-arginine can be used in the pharmaceutical compositions of the invention to enhance delivery of agents to brain tissue after introducing a pharmaceutical composition into the blood stream of the subject substantially contemporaneously with a blood flow enhancing amount of L-arginine. WO 00/56328

Inhibitors of ErbB receptor signaling of the invention, e.g., small chemical compounds, that exert their physiological effect in vivo in the brain can be more useful if they gain access to target cells in the brain. Non-limiting examples of brain cells are neurons, glial cells (astrocytes, oligodendrocytes, microglia), cerebrovascular cells (muscle cells, endothelial cells), and cells that comprise the meninges. The blood brain barrier (“BBB”) typically restricts access to brain cells by acting as a physical and functional blockade that separates the brain parenchyma from the systemic circulation (see, e.g., Pardridge et al., J. Neurovirol. 5: 556-69, 1999; Rubin et al., Rev. Neurosci. 22: 11-28, 1999). Circulating molecules are normally able to gain access to brain cells via one of two processes: lipid-mediated transport through the BBB by free diffusion, or active (or catalyzed) transport.

Inhibitors of ErbB receptor signaling of the invention, e.g., small chemical compounds can be formulated to improve distribution in vivo, for example as powdered or liquid tablet or solution for oral administration or as a nasal spray, nose drops, a gel or ointment, through a tube or catheter, by syringe, by packtail, by pledget, or by submucosal infusion. For example, the blood-brain barrier (BBB) excludes many highly hydrophilic agents. To ensure that the more hydrophilic therapeutic agents of the invention cross the BBB, they can be formulated, for example, in liposomes. For methods of manufacturing liposomes, (see, e.g., U.S. Pat. Nos. 4,522,811; 5,374,548; and 5,399,331). The liposomes can comprise one or more moieties which are selectively transported into specific cells or organs (“targeting moieties” or “targeting groups” or “transporting vectors”), thus providing targeted drug delivery (see, e.g., Ranade, J. Clin. Pharmacol. 29: 685, 1989). Likewise, the agents can be linked to targeting groups that facilitate penetration of the blood brain barrier. In one embodiment, the method of the present invention employs a naturally occurring polyamine linked to an agent that is a small molecule and is useful for inhibiting Aβ deposition.

To facilitate transport of agents of the invention across the BBB, they can be coupled to a BBB transport vector (for review of BBB transport vectors and mechanisms, see, Bickel et al., Adv. Drug Delivery Reviews 46: 247-79, 2001). Exemplary transport vectors include cationized albumin or the OX26 monoclonal antibody to the transferrin receptor; these proteins undergo absorptive-mediated and receptor-mediated transcytosis through the BBB, respectively. Natural cell metabolites that can be used as targeting groups, include, inter alia, putrescine, spermidine, spermine, or DHA. Other exemplary targeting moieties include folate or biotin (see, e.g., U.S. Pat. No. 5,416,016); mannosides (Umezawa et al., Biochem. Biophys. Res. Commun. 153: 1038, 1988); antibodies (P. G. Bloeman, et al., FEBS Lett. 357: 140, 1995); M. Owais, et al., Antimicrob. Agents Chemother. 39: 180, 1995); surfactant protein A receptor (Briscoe et al., Am. J. Physiol. 1233: 134, 1995); gp120 (Schreier et al., J. Biol. Chem. 269: 9090, 1994; see also, K. Keinanen and M. L. Laukkanen, FEBS Lett. 346: 123, 1994; J. J. Killion and I. J. Fidler, Immunomethods 4: 273, 1994).

Examples of other BBB transport vectors that target receptor-mediated transport systems into the brain include factors such as insulin, insulin-like growth factors (“IGF-I,” and “IGF-II”), angiotensin II, atrial and brain natriuretic peptide (“ANP,” and “BNP”), interleukin I (“IL-1”) and transferrin. Monoclonal antibodies to the receptors that bind these factors can also be used as BBB transport vectors. BBB transport vectors targeting mechanisms for absorptive-mediated transcytosis include cationic moieties such as cationized LDL, albumin or horseradish peroxidase coupled with polylysine, cationized albumin or cationized immunoglobulins. Small basic oligopeptides such as the dynorphin analogue E-2078 and the ACTH analogue ebiratide can also cross the brain via absorptive-mediated transcytosis and are potential transport vectors.

Other BBB transport vectors target systems for transporting nutrients into the brain. Examples of such BBB transport vectors include hexose moieties, e.g., glucose and monocarboxylic acids, e.g., lactic acid and neutral amino acids, e.g., phenylalanine and amines, e.g., choline and basic amino acids, e.g., arginine, nucleosides, e.g., adenosine and purine bases, e.g., adenine, and thyroid hormone, e.g., triiodothyridine. Antibodies to the extracellular domain of nutrient transporters can also be used as transport vectors. Other possible vectors include angiotensin II and ANP, which can be involved in regulating BBB permeability.

In some cases, the bond linking the therapeutic agent to the transport vector can be cleaved following transport into the brain in order to liberate the biologically active agent. Exemplary linkers include disulfide bonds, ester-based linkages, thioether linkages, amide bonds, acid-labile linkages, and Schiff base linkages. Avidin/biotin linkers, in which avidin is covalently coupled to the BBB drug transport vector, can also be used. Avidin itself can be a drug transport vector.

Transcytosis, including receptor-mediated transport of compositions across the blood brain barrier, can also be suitable for the agents of the invention. Transferrin receptor-mediated delivery is disclosed in U.S. Pat. Nos. 5,672,683; 5,383,988; 5,527,527; 5,977,307; and 6,015,555. Transferrin-mediated transport is also known. (P. M. Friden et al., Pharmacol. Exp. Ther. 278: 1491-98, 1996; H. J. Lee, J. Pharmacol. Exp. Ther. 292: 1048-52, 2000). EGF receptor-mediated delivery is disclosed in (Y. Deguchi et al., Bioconjug. Chem. 10: 32-37, 1999), and transcytosis is described in (A. Cerletti et al., J. Drug Target. 8: 435-46, 2000). Insulin fragments have also been used as carriers for delivery across the blood brain barrier. (M. Fukuta et al., Pharm. Res. 11: 1681-88, 1994). Delivery of agents via a conjugate of neutral avidin and cationized human albumin has also been described. (Y. S. Kang, et al., Pharm. Res. 1: 1257-64, 1994).

Other modifications in order to enhance penetration of the agents of the invention across the blood brain barrier can be accomplished using methods and derivatives known in the art. For example, U.S. Pat. No. 6,024,977 discloses covalent polar lipid conjugates for targeting to brain and central nervous system. U.S. Pat. No. 5,017,566 discloses cyclodextrin derivatives comprising inclusion complexes of lipoidal forms of dihydropyridine redox targeting moieties. U.S. Pat. No. 5,023,252 discloses the use of pharmaceutical compositions comprising a neurologically active drug and a compound for facilitating transport of the drug across the blood-brain barrier including a macrocyclic ester, diester, amide, diamide, amidine, diamidine, thioester, dithioester, thioamide, ketone or lactone. U.S. Pat. No. 5,024,998 discloses parenteral solutions of aqueous-insoluble drugs with cyclodextrin derivatives. U.S. Pat. No. 5,039,794 discloses the use of a metastatic tumor-derived egress factor for facilitating the transport of compounds across the blood-brain barrier. U.S. Pat. No. 5,112,863 discloses the use of N-acyl amino acid derivatives as antipsychotic drugs for delivery across the blood-brain barrier. U.S. Pat. No. 5,124,146 discloses a method for delivery of therapeutic agents across the blood-brain barrier at sites of increase permeability associated with brain lesions. U.S. Pat. No. 5,153,179 discloses acylated glycerol and derivatives for use in a medicament for improved penetration of cell membranes. U.S. Pat. No. 5,177,064 discloses the use of lipoidal phosphonate derivatives of nucleoside antiviral agents for delivery across the blood-brain barrier. U.S. Pat. No. 5,254,342 discloses receptor-mediated transcytosis of the blood-brain barrier using the transferrin receptor in combination with pharmaceutical compounds that enhance or accelerate this process. U.S. Pat. No. 5,258,402 discloses treatment of epilepsy with imidate derivatives of anticonvulsive sulfamate. U.S. Pat. No. 5,270,312 discloses substituted piperazines as central nervous system agents. U.S. Pat. No. 5,284,876 discloses fatty acid conjugates of dopamine drugs. U.S. Pat. No. 5,389,623 discloses the use of lipid dihydropyridine derivatives of anti-inflammatory steroids or steroid sex hormones for delivery across the blood-brain barrier. U.S. Pat. No. 5,405,834 discloses prodrug derivatives of thyrotropin releasing hormone. U.S. Pat. No. 5,413,996 discloses acyloxyalkyl phosphonate conjugates of neurologically-active drugs for anionic sequestration of such drugs in brain tissue. U.S. Pat. No. 5,434,137 discloses methods for the selective opening of abnormal brain tissue capillaries using bradykinin infused into the carotid artery. U.S. Pat. No. 5,442,043 discloses a peptide conjugate between a peptide having a biological activity and incapable of crossing the blood-brain barrier and a peptide which exhibits no biological activity and is capable of passing the blood-brain barrier by receptor-mediated endocytosis. U.S. Pat. No. 5,466,683 discloses water soluble analogues of an anticonvulsant for the treatment of epilepsy. U.S. Pat. No. 5,525,727 discloses compositions for differential uptake and retention in brain tissue comprising a conjugate of a narcotic analgesic and agonists and antagonists thereof with a lipid form of dihydropyridine that forms a redox salt upon uptake across the blood-brain barrier that prevents partitioning back to the systemic circulation.

Still further examples of modifications that enhance penetration of the blood brain barrier are described in PCT Application Publication Number WO 85/02342, which discloses a drug composition comprising a glycerolipid or derivative thereof. PCT Publication Number WO 089/11299 discloses a chemical conjugate of an antibody with an enzyme which is delivered specifically to a brain lesion site for activating a separately-administered neurologically-active prodrug. PCT Publication Number WO 91/04014 discloses methods for delivering therapeutic and diagnostic agents across the blood-brain barrier by encapsulating the drugs in liposomes targeted to brain tissue using transport-specific receptor ligands or antibodies. PCT Publication Number WO 91/04745 discloses transport across the blood-brain barrier using cell adhesion molecules and fragments thereof to increase the permeability of tight junctions in vascular endothelium. PCT Publication Number WO 91/14438 discloses the use of a modified, chimeric monoclonal antibody for facilitating transport of substances across the blood-brain barrier. PCT Publication Number WO 94/01131 discloses lipidized proteins, including antibodies. PCT Publication Number WO 94/03424 discloses the use of amino acid derivatives as drug conjugates for facilitating transport across the blood-brain barrier. PCT Publication Number WO 94/06450 discloses conjugates of neurologically-active drugs with a dihydropyridine-type redox targeting moiety and comprising an amino acid linkage and an aliphatic residue. PCT Publication Number WO 94/02178 discloses antibody-targeted liposomes for delivery across the blood-brain barrier. PCT Publication Number WO 95/07092 discloses the use of drug-growth factor conjugates for delivering drugs across the blood-brain barrier. PCT Publication Number WO 96/00537 discloses polymeric microspheres as injectable drug-delivery vehicles for delivering bioactive agents to sites within the central nervous system. PCT Publication Number WO 96/04001 discloses omega-3-fatty acid conjugates of neurologically-active drugs for brain tissue delivery. PCT WO 96/22303 discloses fatty acid and glycerolipid conjugates of neurologically-active drugs for brain tissue delivery.

In general, it is well within the ordinary skill in the art to prepare an ester, amide or hydrazide derivative of an agent of the invention, for example, from the corresponding carboxylic acid and a suitable reagent. For instance, a carboxylic acid-containing compound, or a reactive equivalent thereof, can be reacted with a hydroxyl-containing compound, or a reactive equivalent thereof, so as to provide the corresponding ester. (See, e.g., COMPREHENSIVE ORGANIC TRANSFORMATIONS, 2^{nd} Ed., by R. C. Larock, VCH Publishers John Wiley & Sons, Ltd., 1989; MARCH'S ADVANCED ORGANIC CHEMISTRY, 5^{th} Ed., by M. B. Smith and J. March, John Wiley & Sons, Ltd. (2000).

### Formulation

Small chemical molecule, siRNA molecule, or antibody inhibitors of ErbB receptor, dopamine receptor, glutamate receptor, or NRG-1, nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of a psychiatric disorder, are often administered as pharmaceutical compositions comprising an active therapeutic agent, i.e., and a variety of other pharmaceutically acceptable components. See Remington's Pharmaceutical Science (15^{th} ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose), polymeric amino acids, amino acid copolymers, and lipid aggregates (such as oil droplets or liposomes). Additionally, these carriers can function as immunostimulating agents (i.e., adjuvants).

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

### Toxicity

Preferably, a therapeutically effective dose of the small chemical molecule, siRNA molecule, antibody, or nucleic acid compositions, e.g., antisense oligonucleotides, double stranded RNA oligonucleotides (RNAi), or DNA oligonucleotides (vectors) containing nucleotide sequences encoding for the transcription of shRNA molecules, described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1,

### Kits

Also within the scope of the invention are kits comprising the small chemical molecule, siRNA molecule, antibody, or nucleic acid compositions, e.g., antisense oligonucleotides, double stranded RNA oligonucleotides (RNAi), or DNA oligonucleotides (vectors) containing nucleotide sequences encoding for the transcription of shRNA molecules) of the invention and instructions for use. The kit can further contain a least one additional reagent, or one or more additional human antibodies (e.g., a human antibody having a complementary activity which binds to an epitope in the antigen distinct from the first human antibody). Kits typically include a label indicating the intended use of the contents of the kit. The term label includes any writing, or recorded material supplied on or with the kit, or which otherwise accompanies the kit.

The invention will be further described with reference to the following examples; however, it is to be understood that the invention is not limited to such examples.

### EXEMPLARY EMBODIMENTS

### Example 1

### ErbB Receptor Inhibitors and their Use as Anticancer Agents

The ErbB family of receptor tyrosine kinases (RTKs), comprised of ErbB 1, ErbB 2, ErbB 3, and ErbB 4, recently has been the target for intervention of a variety of cancers (see, for example, Grunwald and Hidalgo, J. Natl. Cancer Inst. 95:851, 2003 Shridhar et al., Lancet Oncol. 4: 397, 2003; Albanell J, Gascon P, Current Drug Targets 6: 259-274, 2005; Shelton et al., Expert Opin. Ther. Targets 9: 1009, 2005). The binding of EGF and other ligands that harbor an “EGF-like” domain enhances the homo- and hetero-dimerization of these type I transmembrane proteins, autophosphorylation of tyrosines, recruitment of adapter protein, and the downstream signaling of numerous pathways which include MAP, PI3 and src kinases. See, for example, Lemke, Mol. Cell Neurosci. 7: 247, 1996; Burden and Yarden, Neuron 18:847, 1997; Buonanno and Fischbach, Curr Opin Neurobiol. 11: 287-289, 2001. Because of the implication of the ErbB signaling pathway in the malignant growth associated with breast cancer, glioblastomas medulloblastomas and non-small cell lung carcinoma, there has been intensive research to identify compounds that inhibit ErbB activity.

The inhibitors developed to date fall into 3 general categories: I) monoclonal antibodies that interact with extracellular epitopes on the receptor to reduce interactions and/or promote internalization from the cell surface, II) proteins that interact with the dimerization motif of ErbB receptors to prevent their association, and III) small organic molecules that target the cytoplasmic tyrosine kinase domain to block autophosphorylation and association of ErbB receptors with downstream signaling pathways. This application emphasizes the use of Category III inhibitors because of their small size and physicochemical properties that are compatible with their systemic use.

### Example 2

### Inhibitors of ErbB Receptors

Category I inhibitors. Humanized monoclonal antibodies are presently in use which target the extracellular domains of ErbB-1 and -2 to promote their internalization and/or interfere with their dimerization. Perhaps the most successful examples are trastuzumab (trade name Herceptin) which is approved for treatment of breast cancers over-expressing ErbB-2, and cetuximab which is used to treat non-small-cell lung cancer (NSCLC); both are in on-going Phase I-IV clinical trials for combination treatments. Other humanized antibodies targeting ErbB-1 and -2 are at different stages of on-going clinical trials.

Category II inhibitor. Herstatin (previously known as Dimercept) is a naturally occurring soluble, truncated extracellular portion of ErbB-2 protein that can bind to ErbB receptors to prevent their dimerization and signaling with other ErbB receptors.

Category III inhibitors. While both Category I and II approaches show promise for treating a variety of cancers in peripheral tissues, their inability to cross the blood-brain barrier present a serious limitation for treating CNS cancers or disorders. In contrast, small, membrane permeable molecules with distinct preferences for ErbB receptors are compatible as systemically delivered compounds that may cross the blood-brain barrier. FIG. 1 shows chemical structure of small chemical ErbB inhibitors presently under clinical trials (from Fry, Exp. Cell Research 284: 131, 2003). A variety of these small organic ErbB inhibitors are presently in different stages of preclinical and clinical trials for the treatment of non-small cell lung carcinomas, adenocarcinoma, breast cancer and glioblastomas (see FIG. 1). Among these inhibitors are gefitinib (ZD1839, Iressa) and erlotinib (Tarceva, CP-358774, OSI 774) which have preferences for ErbB1 receptors, and ones with a broader ErbB receptor specificity which include lapatinib (GW572016), EKB-569, CI-1033 (PD183805), AEE788, PKI-146, and PD158780 (Shridhar et al., Lancet Oncol. 4: 397, 2003; Shelton et al., Expert Opin. Ther. Targets 9: 1009, 2005). This application focuses on the Category III compounds.

### Example 3

### Design and Mechanism of Action of Small Inhibitors

The small molecule inhibitors share common structures that allow their interaction with the ATP pocket in the ErbB receptor tyrosine kinase domain. The identification of 4-anilino-quinazolines as important for the specificity and potency of these molecules, lead to the identification of other chemical templates like pyridopyrimidines (PD158780), pyrrollopyridimidines (EKB569), quinoline-3-carbonitriles, pyrazolopyrimidines and thiazolyquinazolines (GW572016) as structures that could improve potency and selectivity for ErbB receptors (rev. Fry, Exp. Cell Research 284: 131, 2003).

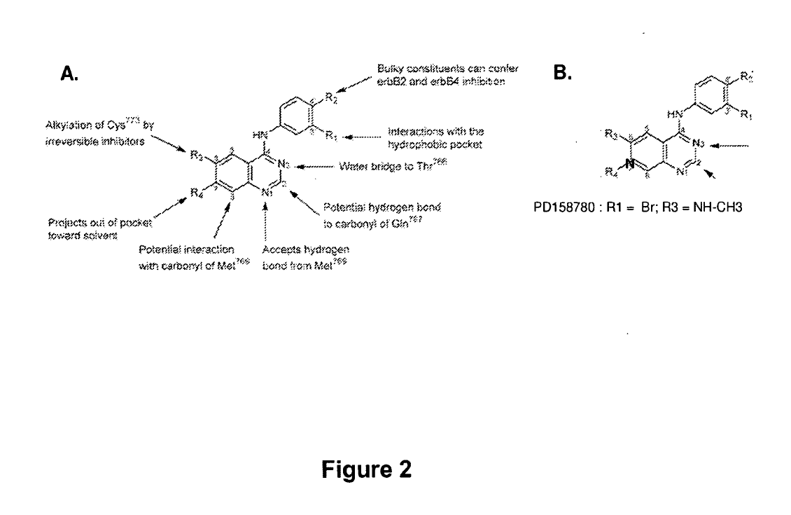

The crystallization of the ErbB-1 extracellular domain with 4-anilinoquinazoline, by Stamos et al. (2002), allowed modeling to improve the design of compounds with different affinities and preferences for ErbB receptors (FIG. 2). FIG. 2 shows core structures of quinalines (A) and pyridopyrimidines (B), and substitution sites for side groups that modify ErbB receptor preferences, affinity and reversibility of inhibitors (Fry, Exp. Cell Research 284: 131, 2003). B) The side groups of PD158780 are indicated. PD168393, in contrast to PD158780, has substitutions at R3 that render it irreversible by alkylation of a Cys located in the ATP pocket of ErbB-1, -2 and -4.

Substitutions on the 4′ position in the aniline with bulky groups lead to favored interactions with ErbB-2, and to a lesser extent ErbB-4 (Fry, Exp. Cell Research 284: 131, 2003; Rusnak et al, Cancer Ther. 1: 85, 2001). One of the high affinity inhibitors generated from modifications to the pyridopyrimidine core is PD158780, a soluble 4-[ar(alk)ylamino]pyridopyrimidine that binds reversibly to ErbB receptors (FIG. 2S,B). PD158780 binds competitively with ATP in the pocket with affinities of 0.1-1 nM in reconstituted receptors in vitro, blocks ErbB phosphorylation in A431, SK-BR-3 and MDA-MB-453 cells with ICs₅₀ of 15-50 nM, and blocks proliferation of these cells at concentration ranging between 0.1-1 μM. Even at μM concentrations no cross-reactivity with other RTKs, such as the PDGF and FGF receptors, was observed (Fry et al., Biochem Pharmacol. 54: 877, 1997). An additional attractive property of PD158780 is that its dissociation rate and autophosphorylation inhibition of the receptors is extremely slow (several hours), thus reducing its clearance time which is problematic with earlier generations of small inhibitors. Another inhibitor is GW572016, a 6-thiazolyquinazoline with IC₅₀ values for ErbB-1, -2 and -4 of 11, 9 and 370 nM, respectively (Fry, Exp. Cell Research 284: 131, 2003; Shridhar et al., Lancet Oncol. 4: 397, 2003).

Irreversible small molecule inhibitors have also been developed by taking advantage of a cysteine (Cys⁷⁷³, Cys⁷⁸⁴ and Cys⁷⁷⁸ in the human ErbB-1, -2 and -4, respectively) located in the ATP binding pocket of ErbB receptors. In the case of 4-anilinoquinazoline cores, the aniline is directed into the hydrophobic pocket of the receptor and the quinazoline is directed towards the ATP pocket where it is in close proximity to the sulfhydryl group of Cys⁷⁷³, as predicted for the crystal structure resolved with Tarseva and ErbB1. The substitution at the 6′ position in quinazolines with mild alkylating agents, or at equivalent positions in other chemical template, foments the formation of a covalent bond making the inhibitors inactivate ErbB receptors irreversibly (FIG. 2S,B). This cysteine is also conserved in ErbB-2 and ErbB-4. Using this principle lead to the development of CI-1033 (also known as PD183805), which has comparable ICs₅₀ between 1-20 nM for the three ErbB receptors (Slichnmyer et al., 2001). PD168393 was synthesized using a similar principle and also binds irreversibly to ErbB receptors. The high affinity and “pan-specificity” of these novel small molecule inhibitors for ErbB have made them attractive for the treatment of numerous tumors that do not necessarily over-express ErbB-2, and preclinical (PD 158780 and 168393) and clinical Phase I and II trials (CI-1033) are presently in progress.

### Example 4

### Feasibility of Using Small ErbB Inhibitors to Treat Psychiatric Disorders

The most common adverse effects of small molecule inhibitors reported during clinical trials are gastrointestinal symptoms, skin rashes and headaches, but in general these compounds are well tolerated when used at their therapeutic concentrations to treat breast or NSCLC (Grunwald and Hidalgo, J. Natl. Cancer Inst. 95:851, 2003; Shridhar et al., Lancet Oncol. 4: 397, 2003; Albanell J, Gascon P Current Drug Targets 6: 259-274, 2005; Shelton et al., Expert Opin. Ther. Targets 9: 1009, 2005). However, it is extremely important to emphasize that the concentrations and regimen of small molecules necessary for the potential use in treatment of psychiatric disorders would be much lower than those utilized for cancer treatment (single doses of 125-1,500 mg, used for several months to years), which need to accumulate high enough to stop and revert malignant growth. The use of irreversible ErbB inhibitors for their potential use in psychiatric disorders, in contrast to cancer treatment, needs to be carefully evaluated because the final goals are different. While irreversible inhibitors are useful as “magic bullets” to permanently terminate transformed cells, the goal of in psychiatric disorders is to modulate ErbB receptor function. Therefore, existing small molecule inhibitors or novel generations of modified quinalines and pyridopyrimidines with “pan-specificity” for ErbB receptors (FIG. 2), potentially could be used for the long-term or episodic treatment of psychiatric disorders with an acceptable incidence of side effects.

### Example 5

### Link Between NRG-1 and the Regulation of Glutamatergic and Dopaminergic Neurotransmission Pathways

The present invention points to a direct link between NRG-1 and the regulation of glutamatergic and dopaminergic neurotransmission pathways. The present findings obtained by using exogenously applied agonist for ErbB (NRG-1 peptide) and dopamine receptors, small molecule inhibitors for both receptors and their downstream signaling pathways, as well as NRG-1 and ErbB-4 mutant mice, show convergent results that implicate a complex NRG/ErbB-glutamate-dopamine signaling network. NRG-1 function may not be limited to glutamatergic and dopaminergic neurotransmission, given the complexity and inter-relationship of different neural networks. It possible that other neurotransmission systems are also affected either directly or indirectly by ErbB receptor activation (i.e., GABAergic, cholinergic transmission). The important issue at this time, however, is that a direct link between NRG/ErbB-glutamate-dopamine signaling has been demonstrated. Our findings are extremely important because these pathways have independently been implicated either genetically (NRG-1) or pharmacologically (glutamate and dopamine receptors) with psychiatric disorders, such as schizophrenia, bipolar disorder, autism and attention deficit disorders. Therefore, our findings open a novel approach for the treatment of psychiatric disorders by either exclusively targeting the ErbB tyrosine kinase pathway with presently available or novel small molecules inhibitors, or by the use of these inhibitors in combination with drugs that modify dopamine and/or glutamate receptor function.

### Example 6

### Role for NRG-1, Glutamatergic Transmission and D2-Type Receptors in the Regulation of Cognitive Processes

Schizophrenia is a major psychiatric disorder affecting approximately 1% of the population. Although in most cases psychosis is manifested during the second decade of life (after puberty), there is an early developmental component to schizophrenia. All known typical and atypical antipsychotics used to treat schizophrenia work either as antagonist or partial agonists on D2-type dopamine receptors. These drugs mostly affect the positive symptoms of schizophrenia (psychosis) but are not as effective at treating the negative symptoms, which are thought to represent the endophenotype of the disorder. The most significant physiological problem with psychosis and cognition in schizophrenia, has been proposed to be the lack of differentiating meaningful from non-meaningful stimuli (or lack of affect) that may result from a general enhancement of cortical activity. Novel strategies and pharmacological agents are necessary to treat both the positive and negative symptoms of this disorder, in particular the cognitive deficits which have the most significant impact on patients lives.

Polymorphisms in the gene encoding Neuregulin-1 (NRG-1), a growth and differentiation factor, recently were associated with a higher inheritance risk for developing schizophrenia. NRGs signal via the family of receptor tyrosine kinases, known as ErbB receptors (ErbB-1 to ErbB-4). Several lines of evidence, utilizing in vitro systems and genetically modified mice, indicate that the NRG signaling pathway regulates the expression of neurotransmitter receptors for glutamate, acetylcholine and GABA in the central nervous system. These 3 neurotransmitter systems have been implicated in schizophrenia, and drug discovery efforts are in progress to target function of these receptors. However, at this moment there is no clear link if, and how, the function of the 3 neurotransmitter systems (Glu, Ach, GABA) are regulated by dopamine, or more specifically, by D2-type dopamine receptors. The present invention provides three major findings that converge to demonstrate a clear function of NRG-1, glutamatergic transmission and D2-type receptors in the regulation of cognitive processes. In essence the present study found that: 1) Neuregulin regulates in a use-dependent fashion the induction and expression of long-term potentiation (LTP); a mechanism believed to underlie memory, learning and cognition. 2) The effects of NRG on LTP are mediated by ErbB receptor tyrosine kinases, more specifically, we found that the ErbB4-type receptor is provides a major role. 3) We identified a cross-talk between NRG/ErbB signaling and dopamine receptor signaling. Distinct selective D2-type receptor antagonists, presently utilized to treat patients with schizophrenia, block the effects of NRGs on LTP. These findings open a novel and exciting approach to developing drugs, or combination therapies, that target both the positive and negative symptoms of schizophrenia. There are several aspects of schizophrenia that are shared with other behavior and cognitive disorders, such as bipolar depression, autism and attention deficit disorder (ADD). In this respect it is interesting that recent genetic studies have also identified an association of NRG-1 polymorphisms with bipolar depression. This opens the possibility that alterations in the NRG-ErbB signaling pathway may contribute to schizophrenia and bipolar depression, as well as other disorders where homeostasis of dopamine neurotransmission have been implicated, such as autism and ADD. For example, genetic variations in mRNAs encoding D4 dopamine receptors have recently been implicated in person diagnosed with ADD, suggesting that modulation of the ErbB signaling pathway could provide novel approaches to address ADD treatment.

### Example 7

### Neuregulin Regulates the Induction and Expression of Long-Term Potentiation

The present study demonstrates that neuregulin regulates in a use-dependent fashion the induction and expression of long-term potentiation (LTP); a mechanism believed to underlie memory, learning and cognition.

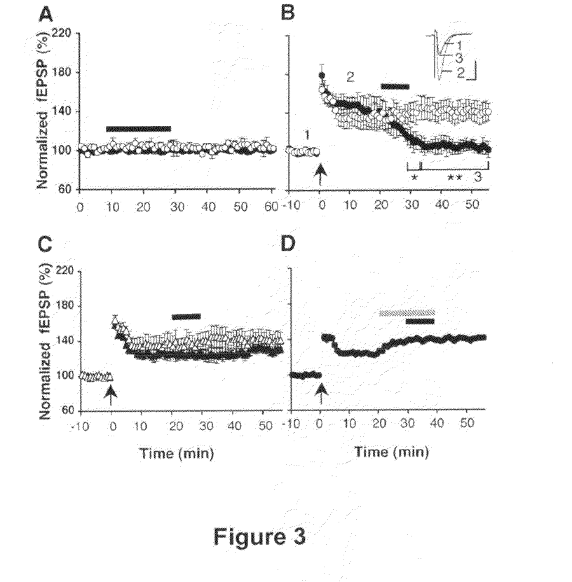

FIG. 3 shows neuregulin-1β regulates LTP depotentiation. While 100 pM NRG-1β (black bar) has no effect on synaptic basal activity (A), it depotentiates LTP (filled circles) at Schaffer collateral-CA1 synapses (B). C) In contrast, NRG-1α (1 nM) has no effect on LTP. D) The effects of NRG-1β are completely blocked by the ErbB RTK inhibitors PD168393 (not shown) and PD158780 (Kwon et al., 2005).

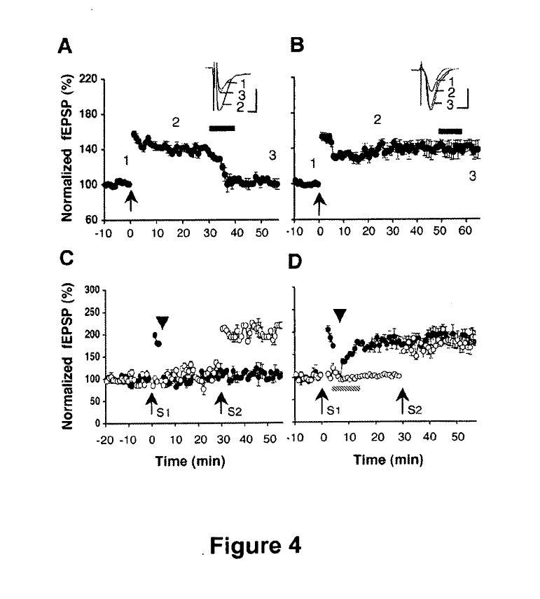

FIG. 4 shows neuregulin-1β effects on LTP depotentiation are time dependent. A and B) NRG-1β (0.1 nM) reverses LTP up to 30 minutes post-induction but has no effect after 50 min. C and D) Using a two-pathway approach, PD158780 was shown to block LTP depotentiation elicited by TPS (Kwon et al., 2005). These results show NRG works in vivo to regulate LTP.

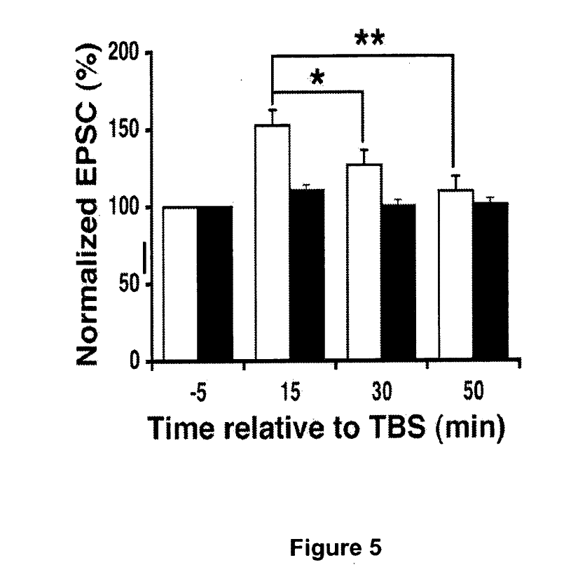

FIG. 5 shows neuregulin-1β selectively downregulates expression of surface AMPA receptors. AMPA and NMDA receptor EPSCs were measured in acute hippocampal slices either 5 min prior (−5) or at 15, 30 and 50 minutes after delivering the theta burst stimulus (TBS) used to elicit LTP. NRG-1β (0.1 nM) was perfused into the chamber between 20 and 30 min following TBS. As observed, AMPA receptor EPSCs increase by ˜50% after TBS, and NRG-1 selectively reverses AMPA (white bars), but not NMDA (black bars), receptor EPSCs.

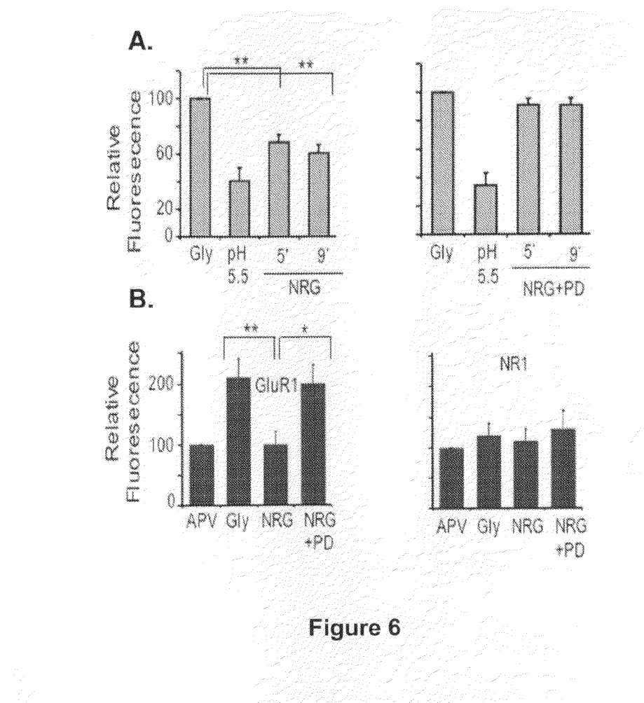

FIG. 6 shows neuregulin-1β causes internalization of surface AMPA receptors in dissociated hippocampal neurons. A) The GluR-1 subunit of AMPA receptors tagged with a fluorescent super ecliptic GFP (seGFP-GluR1), a variant sensitive to pH, was analyzed in transfected neurons. Left, Bathing neurons transiently for ˜1 min with ACSF buffered at pH 5.5 causes a 10 to 20-fold reduction fluorescent levels. Interestingly, incubation with NRG-1 causes a rapid internalization of surface seGFP-GluR1 into endocytic vesicles (internal pH ˜5.0). Right, Internalization of seGFP-GluR1 is completely blocked by the ErbB inhibitor PD 158780. B) Native GluR-1-containing AMPA receptors are internalized by NRG-1 treatment (left), whereas NMDA receptors are not (right).

### Example 8

### ErbB4-Type Receptor Mediates the Effects of NRG on Long Term Potentiation

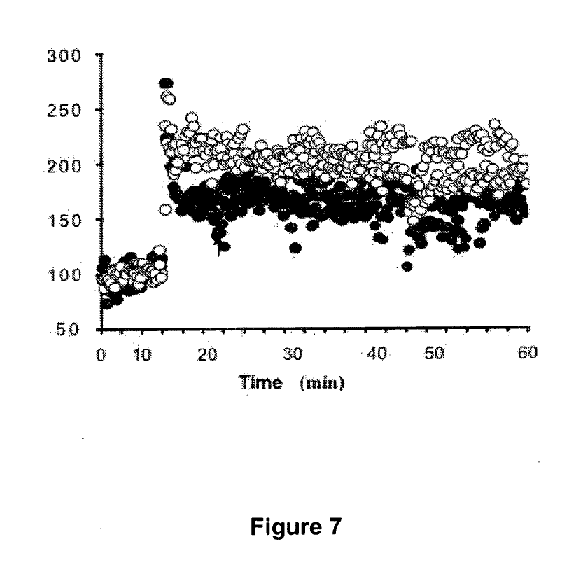

The effects of NRG on LTP are mediated by ErbB receptor tyrosine kinases. More specifically, the ErbB4-type receptor is involved. FIG. 7 shows NRG-1 heterozygote mutant mice have enhanced LTP. Homologous recombination was used to remove the EGF-like active motif from the NRG-1 gene. Mice that are null for the gene die at ˜E9.0 from embryonic heart malformation. Here we show that, consistent with our data showing that exogenously added NRG-1 peptide depotentiates LTP, mice that heterozygotes for the NRG-1 gene (open circles) have higher levels of LTP than wild-type (filled circles) littermates.

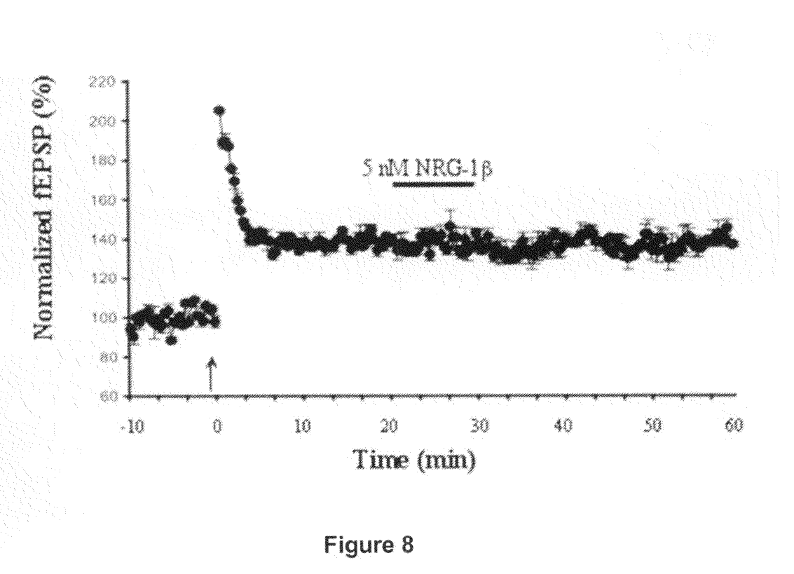

FIG. 8 shows NRG-1 effects on LTP in vivo are selectively mediated by ErbB-4 receptors, as NRG-dependent depotentiation cannot be elicited in ErbB4 knockout mice. Homologous recombination was used to mutate the ErbB-4 receptor gene. Mice that are null for receptor die at ˜E9.5 from embryonic heart malformation, but the mutants were rescued by specific expression of ErbB-4 in heart driven by the α-actin promoter. These results show that NRG-1 effects on synaptic plasticity in the hippocampus do not require ErbB-3, the only other NRG-binding ErbB receptor.

### Example 9

### Dopamine Receptor Signaling Involved in Regulation of Long Term Potentiation

Cross-talk between NRG/ErbB signaling and dopamine receptor signaling has been identified. Distinct selective D2-type receptor antagonists, presently utilized to treat patients with schizophrenia, block the effects of NRGs on LTP.

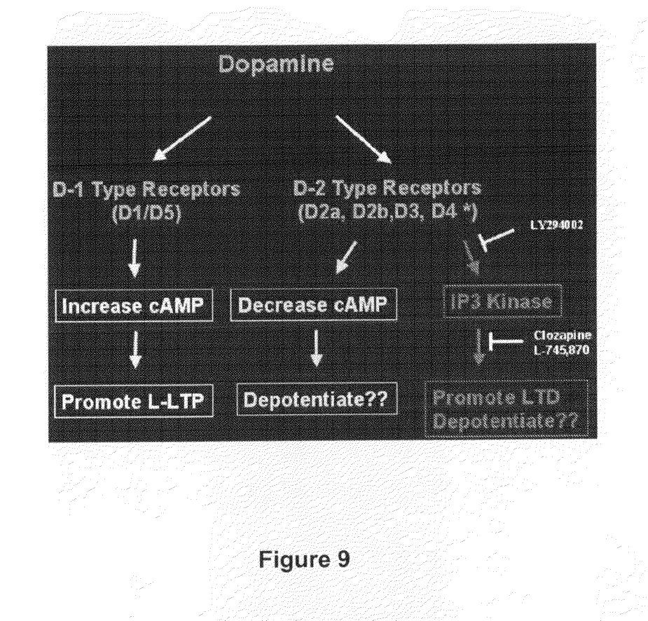

FIG. 9 shows dopamine receptors couple to distinct G-couple pathways and have been implicated in LTP regulation. D2 type receptors (D2, D3 and D4) reduce cAMP levels and also signal via IP3 kinase. We have tested the effects of inhibitors of IP3K (LY294002), D2-type receptors (sulpride) and D4-specific receptors (L745870) on LTP.

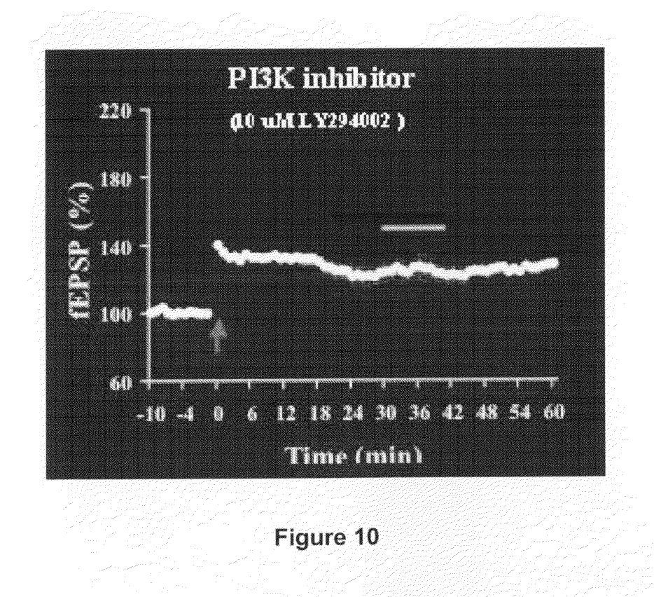

FIG. 10 shows inhibition of the PI3K pathway prevents LTP depotentiation by NRG-1. Slices were perfused with 10 uM LY294002 for 20 min (black line) following LTP induction by TBS (arrow). NRG-1 was added (light blue line) 10 min following the addition of the PI3K inhibitor. As shown, LTP expression was not affected by 5 nM NRG-1; this concentration is 50-fold higher than necessary to produce depotentiation.

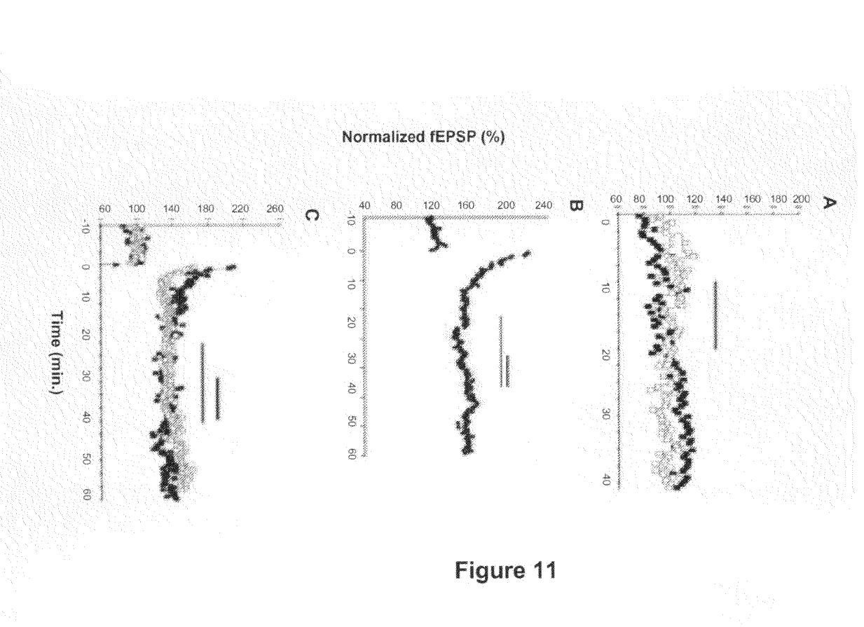

FIG. 11 shows blockade of D2-type receptors prevents NRG-1-dependent LTP depotentiation. A) The D2-selective inhibitors Sulpride or L745870 (blue bar) have no effect on basal synaptic transmission when perfused in hippocampal slices. B) Perfusion with Supride (10 uM), considered a “pan” D2-specific inhibitor, or C) with L745870 (50 nM), a potent D4 receptor antagoinst, prevent NRG-1-evoked LTP depotentiation (filled circles). The open circles (C) are control slices treated with vehicle.

### Example 10

### Screening for Modulators of NRG/ErbB Signaling by Measuring Gamma Oscillatory Activity in Vitro Evoked by Kainate Treatment or Carbachol Treatment

Alterations in gamma frequency rhythmic activity are associated with psychiatric disorders, and polymorphisms in Neuregulin-1 and ErbB4, a Neuregulin-1 receptor, are associated with schizophrenia. The synchronization of neuronal network activity in the human cortex and hippocampus at gamma frequencies, measured from scalp-attached electroencephalogram (EEG) recordings, is important for cognition and memory (A. K. Engel, et al., Trends Cogn Sci 5: 16, 2001. Gamma band activity is altered during psychotic states associated with psychiatric and neurodegenerative disorders. K. M. Spencer et al., Proc Natl Acad Sci USA 101: 17288, 2004; C. S. Herrmann et al., Clin Neurophysiol 116: 2719, 2005. Similar gamma rhythms can be evoked in rodent acute hippocampal slices by bath application of carbachol or kainic acid. A. Fisahn, et al., Nature 394: 186, 1998; S. G. Hormuzdi et al., Neuron 31: 487, 2001; A. Fisahn et al., J Neurosci. 24: 9658, 2004. Spontaneous long-lasting gamma oscillations have been recorded in vivo in the hippocampus and their frequency is modulated by GABAergic basket cell interneurons. A. Fisahn, et al., Nature 394: 186, 1998; A. Bragin et al., J Neurosci 15: 47, 1995; J. Csicsvari et al., Neuron 37: 311, 2003; E. O. Mann et al., Neuron 45: 105, 2005. Recent studies indicate that the power of gamma oscillations in subjects diagnosed with schizophrenia is altered (J. S. Kwon et al., Arch Gen Psychiatry 56: 1001 (November 1999); T. W. Wilson et al., Cereb Cortex, (Jun. 8, 2007)), and is correlated with the positive or negative symptoms of the disorder. K. M. Spencer et al., Proc Natl Acad Sci USA 101: 17288, 2004. Gamma oscillatory activity is regulated by GABAergic interneurons, in particular the parvalbumin-expressing subclass of interneurons. E. C. Fuchs et al., Neuron 53: 591, 2007; M. Vreugdenhil et al., J Neurophysiol 89: 1414, 2003. This subtype of GABAergic interneurons express the ErbB4 receptor at high levels and are found to be decreased selectively in the postmortem brain of individuals with schizophrenia. Z. J. Zhang et al., Schizophr Res 55: 1, 2002; D. A. Lewis et al., Nat Rev Neurosci 6: 312, 2005.

Subjects harboring this “at-risk” NRG-1 polymorphism manifest decreased frontal and temporal lobe activation, decreased IQ and augmented psychotic symptoms (J. Hall et al., Nat Neurosci Oct. 29, 2006. Using a novel biochemical approach that analyzes protein interactions and modifications in membrane fractions isolated from unfixed postmortem brain, Neuregulin-ErbB4 signaling was also reported to be altered in schizophrenia. C. G. Hahn et al., Nat Med 12: 824, 2006. Given the fact that NRG-1 and ErbB4 hypomorphic mice exhibit altered behaviors that are also observed in other schizophrenia animal models, and which are ameliorated by treatment with the antipsychotic clozapine (H. Stefansson et al., Am J Hum Genet 71 (Jul. 23, 2002)), then it is plausible that gamma oscillation frequency or power can be modified by NRG-ErbB signaling. Therefore, methods to measure gamma oscillatory activity in vitro using brain slices, evoked by either kainate or carbachol treatment provide an experimental system to test and screen for modulators of the NRG/ErbB signaling, fast neurotransmission (i.e., glutamatergic, GABAergic, cholinergic) and slower neuromodulators such as monoamines (i.e., dopamine, serotonin, norepinephrine). A. Fisahn, et al., Nature 394: 186, 1998; S. G. Hormuzdi et al., Neuron 31: 487, 2001; A. Fisahn et al., J Neurosci. 24: 9658, 2004.

### Example 11

### Materials and Methods

Peptides corresponding to the human NRG-1β1 (aa 176-246) and NRG-1a2 (aa 177-241) EGF-like domains were bacterially produced and purified to 95% homogeneity (R&D Research, Minneapolis, USA). ErbB receptor inhibitors PD158780 (4-[(3-Bromophenyl)amino]-6-(methylamino)-pyrido[3,4-d]pyridimine) and PD168393 (4-[3(Bromophenyl)amino]-6-acrylamidoquinazoline) were purchased from Calbiochem (San Diego, USA). Inhibitors and NRG-1 peptides were analyzed in ErbB tyrosine phosphorylation assays to confirm their bioactivity. Antibodies: Commercial antisera raised against the extracellular amino terminal domains of GluR1 (rabbit polyclonal) and NR1 (mouse monoclonal) were purchased from Calbiochem (San Diego, USA) and Pharmingen (San Diego, USA), respectively. Secondary antibodies used were Cy3-conjugated goat anti-rabbit (1:600) and Alexa Fluor 488 goat anti-mouse (1:250) from Molecular Probes (Eugene, USA). Animals: C57BL/6 mice used to generate acute slices and Sprague-Dawley rat pups used to make dissociated hippocampal neuronal cultures were purchased from Charles River (Wilmington, USA). Prenatal animals were removed by Caesarian section after the mother had been killed by CO₂ and decapitation. Animals were maintained and sacrificed in accordance with the National Institutes of Health guidelines.

Acute mouse hippocampal slice preparation: Hippocampal slices from 4 to 5 week old C57BL/6 mice were prepared in ice-cold artificial cerebrospinal fluid (ACSF) and cut on a 1000PLUS Vibratome (VIBRATOME, St. Louis, USA) to a thickness of ˜300 μm. Slices were incubated in a holding chamber at 34° C. for 10 min in ACSF saturated with Carbogen (95% O₂-5% CO₂), transferred to room temperature ACSF and kept for at least 1 hr at room temperature before recording. The composition of ACSF was (mM) 125 NaCl, 2.5 KCl, 1 MgCl₂, 2 CaCl₂, 25 glucose, 1.25 NaH₂PO₄, 0.4 ascorbic acid, 3 myo-inositol, 2 sodium pyruvate, 25 NaHCO₃ (pH 7.2-7.4), as described previously (A. K. Engel et al., Trends Cogn Sci 5: 16 (Jan. 1, 2001)).

Electrophysiological recordings in mouse hippocampal slices: A single slice was transferred to a submerged recording chamber continuously perfused at a flow rate of 2 mL/min (30° C.) with recording ACSF (same as cutting ACSF minus the ascorbate, myo-inositol and pyruvate). Recordings were performed in 10 μM bicuculline (Tocris, USA) to block inhibitory activity. Field and whole-cell patch clamp recordings were performed using glass microelectrodes (6-7 MΩ) filled with internal solution (mM: 145 K-gluconate, 10 HEPES, 5 ATP-Mg, 0.2 GTP-Na, 10 BAPTA at pH 7.2 using KOH) and placed in stratum radiatum using infrared microscopy (Nikon Eclipse E600FN, Japan). A borosilicate two-barrel stimulation electrode (World Precision Instruments, Sarasota, Fla.) with a silver wire (A-M Systems, Carlsberg, Wash.) was filled with oxygenated ACSF recording solution and placed in the stratum radiatum to stimulate the Schaffer collateral/commissural fibers at 0.05 Hz (0.1 msec, 20-40 μA) via a stimulus isolator (A365, World Precision Instruments, Sarasota, Fla.). Data were collected with a Multiclamp 700A amplifier (Axon Instrument, Foster city, CA). The recordings were digitized at a sampling rate of 10 kHz and filtered at 3 kHz. Baseline fEPSP slopes were set to 40-50% of maximum response. Baseline recordings of at least 10 min were obtained once stable basal transmission was observed. LTP was induced using a theta burst stimulation (TBS) protocol that consisted of 5 trains delivered at 0.2 Hz. Each train comprised of 5 stimuli 0.1 msec at 100 Hz, as described previously (K. M. Spencer et al., Proc Natl Acad Sci USA 101: page 17288 (Dec. 7, 2004).). Slices that showed less that 130% LTP were not included in the analysis. To measure the AMPAR or the NMDAR components of evoked EPSCs, voltage-clamp recordings from CA1 pyramidal neurons were performed at holding potentials of −70 mV to measure the AMPAR component (10 EPSCs during 1 min) or at +40 mV to measure the NMDAR component. The AMPAR or NMDAR mediated EPSCs were measured 5 min prior to delivering the TBS, at approximately 20 min after inducing LTP (when the response stabilized), and at 10 and 40 min after commencing NRG-1β1 perfusion. In other experiments NMDAR EPSCs were recorded in potentiated slices prior to and after NRG-1 treatment at +40 mV (plus 10 mM CNQX) to inhibit residual AMPAR currents. Paired-pulse facilitation was analyzed in whole-cell current-clamp recordings with inter-pulse intervals set to 50, 100, 150 and 200 msec.

Peptides corresponding to the splice variants of the EGF-like domains of NRG-1β1 and NRG-1α2 were dissolved in recording ACSF solution to a final concentration 0.1 or 1 nM. NRG was delivered for 10 min in external recording ACSF solution 20, 30 or 50 min following TBS. The ErbB receptor inhibitors PD158780 (10 μM) and PD168393 (10 μM) were diluted in ACSF solution and applied to the slices 10 min prior to NRG-1 addition to ensure diffusion into the cell and efficient receptor inhibition. Control solutions of ACSF containing either 0.1% BSA (used to stabilize NRG-1 stocks) or 0.0001% DMSO vehicle (used to dissolve ErbB receptor inhibitors) had no measurable effects on basal synaptic transmission or LTP. Data were analyzed for significance using the Student's t-test. All results are presented as mean±SEM.

Dissociated hippocampal cultures and chemLTP induction: Hippocampal neuronal cultures from E19 rat pups were prepared as reported previously. C. S. Herrmann et al., Clin Neurophysiol 116: 2719, 2005; A. Fisahn, et al., Nature 394: 186, 1998. Neurons were plated on 12 mm glass coverslips in 24 well plates at a density of 50,000 cells/well. ChemLTP was induced in cultures essential as described previously with minor modifications (Liao et al., 2001; Lu et al., 2001). Briefly, cells grown for 5 days in vitro (DIV5) were switched to medium containing 200 μM APV and maintained in Neurobasal media supplemented with B-27 (Invitrogen) until DIV18 to DIV22. To induce chemLTP, cultures were switched for 20 min to 200 μM glycine in ACSF (mM: 125 NaCl, 2.5 KCl, 26.2 NaHCO₃, 1 NaH₂PO₄, 11 glucose, 2.5 CaCl₂), washed in ACSF, followed by an additional 30 min incubation at 37° C. in 5% CO₂ to allow chemLTP to stabilize at maximal levels. Control cultures were maintained in media containing 200 μM APV during the entire experiment.

Live imaging in transfected hippocampal neurons of surface seGFP-tagged GluR1 AMPARs. Hippocampal neurons (DIV 10-12) grown on 25 mm coverslips were co-transfected using Lipofectime 2000 (Invitrogen) with expression constructs encoding GluR1 tagged with the pH sensitive se-GFP and wild-type GluR2. Life imaging of cultures, which had been grown on APV since DIV 5 (see above), began 24-72 h post-transfection. Cultures were transferred to Hepes-buffered balanced salt solution, (HBBSS (mM): 25 Hepes, pH 7.4, 119 NaCl, 2.5 KCl, 10 NaH₂PO₄, 2 CaCl₂, 30 Glucose). Coverslips were loaded into open chambers supported by a PDMI platform (Harvard Apparatus). Cells were perfused with 200 mM glycine-containing medium used to induce chemLTP prior to capturing images and recorded for 4 min. Then, the cells were continuously perfused with HBBSS (pH 7.4) containing either NRG-1 (1 nM, 10 min) or NRG-1+PD158780 (5 min perfusion with PD158780, followed by 10 min PD158780+NRG); 200 μM glycine was present at all times. Brief washes (2.5 min) with acidic MES buffer (mM: 25 MES, pH, 5.5, 119 NaCl, 2.5 KCl, 2 CaCl₂, 30 glucose) were used to quench surface GluR1-seGFP receptors to estimate the proportion of surface receptors. Immediately after quenching with acidic MES buffer, cultures were switched back to 200 μM glycine in HBBSS (pH 7.4) for 2 min to recover the fluorescence. Solution exchanges were performed using a multi-barreled pencil connected to a bank of programmable pinching valves (Automate Scientific), at a flow rate of 0.4 ml/min. Both the PDMI platform and a lens heater (Bioptechs) were used to maintain the temperature at 35° C. Images were collected every 5 seconds using an Ultraview RS spinning disk scanhead (Perkin Elmer) coupled to a Zeiss Axiovert 200M microscope (63X oil immersion lens, Plan-Apo, NA 1.4) and an Argon-Krypton laser (15 mW, 488 excitation line). Images were detected with a cooled CCD camera (10 mW output, 12 bits, 1344×1024 pixels, Hamamatsu Orca ER) using 400 msec of exposure and 2×2 binning. Fluorescence was determined in 20-μm segments of neuronal processes that were at least 10 μm removed from the neuronal soma. Measurements in the regions of interest were obtained over time using ImageJ. Values represent the means of 6 (NRG-1) and 5 (NRG-1+PD) experiments±SEM. Significance was evaluated by 1-way-ANOVA.

Immunocytochemical analysis of surface AMPARs and NMDARs in chemLTP and NRG-1-treated cultures. To study the effect of NRG-1 on chemLTP, cells were switched from the 200 μM APV culture media to ACSF containing 200 μM glycine and plus 0.5 nM NRG-101 with or without the ErbB receptor inhibitor PD158780 (10 μM) for 20 min; cultures were pretreated with the inhibitor for 10 min prior to NRG-1 addition. All incubations and washes were performed at 37° C. in 5% CO₂. Upon completion of the treatments, control (APV only), chemLTP (glycine only), NRG-1 and NRG-1+PD158780 cultures were fixed with 4% PFA-PBS for 20 min. After 3 washes with PBS, cells were blocked with 10% normal goat serum-PBS (NGS-PBS), and AMPARs and NMDARs were labeled overnight at 4° C. in 2% NGS-PBS with antibodies raised against the extracellular domain of GluR1 (1:400) and NR1 (1:250) under non-permeabilizing conditions. Secondary goat anti-mouse (conjugated to Alexa 488) and goat anti-rabbit (conjugated to Cy3) antibodies were applied for 1 hour at room temperature. Optical images were collected with dual channels for Cy3 and A488 fluorescence using a Zeiss LSM-500 with a 100× oil objective lens (NA 1.4). Total fluorescence in areas of 25 μm of dendrites (ROI) from 10 neurons/treatment for each experiment (n=4) were analyzed with Metamorph Imaging software; cell bodies and proximal segments of dendrites were excluded. Data from 70-80 ROI were collected for each treatment for each experiment, and normalized relative to the values for APV (set=1). The results are presented as means±SEM (4 experiments). Significance was evaluated by 1-way-ANOVA.

Each recited range includes all combinations and sub-combinations of ranges, as well as specific numerals contained therein.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed:

1. A method for treating a psychiatric disorder in a mammalian subject comprising administering to the subject a modulator of ErbB receptor signaling pathway in an amount effective to reduce or eliminate the psychiatric disorder in the subject or to prevent its occurrence or recurrence.

2. The method of claim 1 wherein the modulator is a small chemical compound, an antisense RNA, an siRNA, shRNA, antibody, peptide or peptide mimetic.

3. The method of claim 1 wherein ErbB receptor signaling occurs by ligand activation.

4. The method of claim 1 wherein the modulator is an inhibitor of ErbB tyrosine kinase or an antagonist of ErbB receptor signaling pathway.

5. The method of claim 1 wherein the modulator is an activator of ErbB tyrosine kinase or an agonist of ErbB receptor signaling pathway.

6. The method of claim 4 wherein the inhibitor or the antagonist modulates induction or expression of long term potentiation in the mammalian subject.

7. The method of claim 5 wherein the activator or the agonist modulates induction or expression of long term potentiation in the mammalian subject.

8. The method of claim 1 wherein the psychiatric disorder is schizophrenia, bipolar depression, autism, or attention deficit disorder.

9. The method of claim 1, wherein the modulator of ErbB receptor signaling is a modulator of dopaminergic transmission signaling.

10. The method of claim 9, wherein the modulator of ErbB receptor signaling affects an activity of dopamine D2 type receptor signaling.

11. The method of claim 9 wherein the modulator of dopaminergic transmission signaling modulates induction or expression of long term potentiation in the mammalian subject.

12. The method of claim 1, wherein the modulator of ErbB receptor signaling is a modulator of one or more ADAM proteases.

13. The method of claim 1, wherein the modulator of ErbB receptor signaling is a modulator of gamma-secretase.

14. The method of claim 1, wherein the modulator of ErbB receptor signaling is a modulator of glutamatergic transmission signaling.

15. The method of claim 1 further comprising administering to the subject a modulator of glutamatergic transmission signaling.

16. The method of claim 1 further comprising administering to the subject a modulator of dopaminergic transmission signaling.

17. The method of claim 1 further comprising administering to the subject a modulator of ADAM protease activity.

18. The method of claim 1 further comprising administering to the subject a modulator of gamma secretase activity.

20. The method of claim 1 wherein the ErbB receptor modulator is a modulator of cholinergic transmission signaling.

21. The method of claim 1 wherein the ErbB receptor is ErbB1 receptor, ErbB2 receptor, ErbB3 receptor, or ErbB4 receptor.

22. The method of claim 21 wherein the ErbB receptor is ErbB4 receptor.

23. The method of claim 3 wherein the ligand is an EGF-like motif-containing ligand.

24. The method of claim 23 wherein the EGF-like motif-containing ligand is neuregulin.

25. The method of claim 24 wherein the neuregulin is neuregulin 1, neuregulin 2, neuregulin 3, or neuregulin 4.

26. A method for identifying a modulator of signaling in cells via an ErbB receptor signaling pathway comprising,
contacting a test compound with a cell-based assay system comprising a cell expressing ErbB receptor capable of signaling responsiveness to a ligand and expressing dopamine receptor capable of signaling responsiveness to dopamine,
providing the ligand to the assay system in an amount selected to be effective to modulate ErbB receptor signaling, and
detecting an effect of the test compound on ErbB receptor signaling in the assay system, effectiveness of the test compound in the assay being indicative of the modulation.

27. The method of claim 26 wherein the ligand is an EGF-like motif-containing ligand.

28. The method of claim 27 wherein the ligand is neuregulin 1, neuregulin 2, neuregulin 3, or neuregulin 4.

29. The method of claim 26 wherein the ErbB receptor is an ErbB1 receptor, ErbB2 receptor, ErbB3 receptor, or ErbB4 receptor.

30. The method of claim 26 wherein the cells are hippocampal cells, neuronal cells, glial cells, brain tissue, olfactory epithelial cells, or neuroepithelial cells.

31. The method of claim 26 wherein the detecting step further comprises measuring theta-pulse stimuli depotentiation of long term potentiation in the cell in response to ligand signaling via ErbB receptor.

32. The method of claim 31 wherein the detecting step further comprises modulating depotentiation of long term potentiation in response to theta pulse stimuli by administration of the compound in the cellular assay.

33. The method of claim 26 wherein the detecting step further comprises measuring dopamine receptor signaling in response to dopamine.

34. The method of claim 33 wherein the detecting step further comprises measuring modulation of dopamine receptor signaling and modulation of long term potentiation in response to theta pulse stimuli by administration of the compound in the cellular assay.

35. The method of claim 26, further comprising screening a compound to treat a a psychiatric disorder in a mammalian subject by detecting an effect of the test compound on ErbB receptor signaling in the assay system, effectiveness of the test compound in the assay being indicative of efficacy of treatment in the mammalian subject.

36. A method for identifying a modulator of signaling in cells via an ErbB receptor signaling pathway comprising,
contacting a test compound with a tissue-based assay system comprising a tissue expressing ErbB receptor capable of signaling responsiveness to a ligand,
providing the ligand to the assay system in an amount selected to be effective to modulate ErbB receptor signaling, and
detecting an effect of the test compound on ErbB receptor signaling in the assay system by measuring a change in amplitude or frequency of gamma oscillatory activity, effectiveness of the test compound in the assay being indicative of the modulation.

37. The method of claim 36 wherein the detecting step further comprises measuring a change in frequency or power of kainate-induced or carbachol-induced gamma oscillatory activity.

38. The method of claim 36 wherein the tissue based assay comprises brain tissue.

39. A method for screening a compound to treat a psychiatric disorder in a mammalian subject comprising,
contacting a test compound with a cell-based assay system comprising a cell expressing ErbB receptor capable of signaling responsiveness to a ligand and expressing D2 type receptor capable of signaling responsiveness to dopamine,
providing the ligand to the assay system in an amount selected to be effective to modulate ErbB receptor signaling, and
detecting an effect of the test compound on ErbB receptor signaling in the assay system, effectiveness of the test compound in the assay being indicative of efficacy of treatment in the mammalian subject.

40. The method of claim 39 wherein the psychiatric disorder is schizophrenia, attention deficit disorder, bipolar depression, or autism.

41. A method for diagnosing a psychiatric disorder in a mammalian subject comprising
isolating lymphocytes expressing NRG gene and ErbB receptor gene,
analyzing splice variants of NRG mRNA or ErbB mRNA, and
determining a predisposition to a psychiatric disorder on the basis of splice variants of NRG mRNA or ErbB mRNA.

42. The method of claim 41, wherein the NRG splice variants encode type I, type II, type III or type IV neuregulin-1.

43. The method of claim 41 wherein the NRG gene encodes neuregulin 1, neuregulin 2, neuregulin 3, or neuregulin 4.

44. The method of claim 41 wherein the ErbB receptor gene encodes ErbB1 receptor, ErbB2 receptor, ErbB3 receptor, or ErbB4 receptor.

45. The method of claim 41 wherein the psychiatric disorder is schizophrenia, attention deficit disorder, bipolar depression, or autism.

46. A method for diagnosing a psychiatric disorder in a mammalian subject comprising
isolating neural cells expressing NRG gene and ErbB receptor gene,
analyzing polymorphisms of the NRG gene or the ErbB receptor gene, and
determining a predisposition to a psychiatric disorder on the basis of polymorphisms of the NRG-1 gene or the ErbB4 receptor gene.

47. The method of claim 46 wherein the polymorphism of NRG gene or ErbB receptor gene is a single nucleotide polymorphism.

48. The method of claim 46 wherein the NRG gene encodes neuregulin 1, neuregulin 2, neuregulin 3, or neuregulin 4.

49. The method of claim 46 wherein the ErbB receptor gene encodes ErbB1 receptor, ErbB2 receptor, ErbB3 receptor, or ErbB4 receptor.

50. The method of claim 46 wherein the psychiatric disorder is schizophrenia, attention deficit disorder, bipolar depression, or autism.

51. The method of claim 46 wherein the neural cell is a hippocampal cell, neuronal cell, glial cell, brain tissue, olfactory epithelial cell, or neuroepithelial cell.
